(19) United States
(12) Patent Application Publication
(10) Pub. No.: US 2026/0146283 A1
(43) Pub. Date: May 28, 2026

(54) METHODS AND COMPOSITIONS FOR DNA LIBRARY PREPARATION AND ANALYSIS

(71) Applicant: Roche Sequencing Solutions, Inc., Pleasanton, CA (US)

(72) Inventors: Jagadeeswaran Chandrasekar, Seattle, WA (US); Joseph Welborn Horsman, Seattle, WA (US); Mark Stamatios Kokoris, Bothell, WA (US); Robert N. McRuer, Mercer Island, WA (US); John C. Tabone, Kirkland, WA (US)

(73) Assignee: Roche Sequencing Solutions, Inc., Pleasanton, CA (US)

(21) Appl. No.: 19/339,854

(22) Filed: Sep. 25, 2025

Related U.S. Application Data
(63) Continuation of application No. PCT/EP2024/057566, filed on Mar. 21, 2024.
(60) Provisional application No. 63/456,367, filed on Mar. 31, 2023.

(51) Int. Cl. C12Q 1/6855

(52) U.S. Cl. CPC C12Q 1/6855

(57) ABSTRACT

Provided are DNA library preparation methods and compositions that duplicate a target nucleic acid sequence. A target DNA template including the target sequence is circularized via an end adapter to form a circular construct, which is bidirectionally extended by a polymerase-mediated extension that is initiated at nick sites of the end adapter. Following polymerase-mediated extension, a double-length DNA template is formed that includes two copies of the target DNA template (and hence two copies of the target sequence). Each strand of the double-length DNA template includes a parental polynucleotide strand joined to a newly synthesized daughter strand copy of the parental polynucleotide strand. Predetermined sequences can be included in the double-length DNA template, such a primer sequences, unique molecule identifiers, and sequence indexes. Sequencing of the double-length DNA template can reveal genetic/epigenetic information associated with the target sequence. Also provided are methods to create asymmetric and multi-length DNA template constructs.

### CROSS REFERENCE TO RELATED APPLICATIONS

This patent application is a continuation of International Patent Application No. PCT/EP2024/057566 filed Mar. 21, 2024, which claims priority to and the benefit of U.S. Provisional Application No. 63/456,367, filed Mar. 31, 2023. Each of the above patent applications is incorporated herein by reference as if set forth in its entirety.

### STATEMENT REGARDING SEQUENCE LISTING

The Sequence Listing associated with this application is provided in xml format in lieu of a paper copy, and is hereby incorporated by reference into the specification. The name of the text file containing the Sequence Listing is P37440-US-1_Sequence_Listing.xml. The xml file is 4,506 bytes, and was created on Sep. 23, 2025.

### FIELD OF THE INVENTION

The present invention relates generally to methods and compositions for preparing a DNA library, and more particularly to methods and compositions for replicating a target DNA template and analyzing the replicated target DNA template for genetic and/or epigenetic information.

### BACKGROUND

Nucleic acid sequencing is a critical technology for biology and medicine. While conventional polymerase chain reaction (PCR) techniques have been highly useful and effective, the required heating and cooling cycles of PCR limit its utility. For example, melting hybridized DNA during the heating cycle can degrade the target sample. Nor are such heating/cooling cycles compatible with investigation of living systems. Because of the limitations with conventional PCT techniques, much investigation has centered on identifying isothermal approaches to nucleic acid amplification.

One conventional isothermal method for producing multiple copies of a target nucleic acid includes Rolling Circle Amplification (RCA), in which a small circular oligonucleotide provides a template for polymerase attachment and unidirectional replication. RCA creates a long, single ss-DNA product that is composed of many sequentially linked (tandem) copies of the target DNA molecule's complement. The method circularizes the target DNA, and initiates polymerase extension with a primer. After replicating around the circularized DNA, the primer is displaced, and the polymerase proceeds on multiple additional rounds of the target DNA creating multiple copies until a termination event occurs. This results in a long, single-stranded DNA strand with several copies of the target. The single-stranded DNA strand can then be read and analyzed.

Single read accuracy for single DNA molecule sequencing, however, has often had limited accuracy. Some techniques to improve accuracy are to i) re-read a molecule, ii) read its complement or iii) read multiple copies of the DNA molecule (e.g., as employed with RCA). For example, a molecule may be read many times by circularizing the target DNA (including both complementary strands) and measuring it multiple times as it loops around a sensing location. Other systems “peel” off the complement strand as it reads one strand and then capture the complement a fraction of the time for reading immediately afterwards. DNA-based universal molecular identifiers (UMI) and sample identifiers (SID) are then spliced into individual molecules prior to PCR amplification so that a measured subset of the family of the resultant amplicon copies can be attributed to a single parent molecule from a specific sample. Reading multiple copies within a family improves the accuracy to which that molecule's sequence is known.

While the above methods are useful, what is needed are methods and compositions that consistently, reliably, and easily replicate a target sequence, such as for library preparation, while controlling the size of the replicate. For example, what are needed are methods and compositions that can duplicate a target sequence, thereby beneficially controlling the size of the template library. What are also needed are methods that replicate both strands of a target sequence while preserving the parental strands in the replicate, thereby facilitating bioinformatic analysis of the target sequence.

### SUMMARY OF THE INVENTION

In certain example aspects, provided is a linear end adapter (EA) for duplicating a linear target DNA template. The EA includes, for example, a first polynucleotide strand hybridized to a second polynucleotide strand, thereby forming polynucleotide duplex. The polynucleotide duplex includes, for example, a first terminal end and a second terminal end. The EA also includes a first nick site and second nick site, the first nick site being located within the first polynucleotide strand of the polynucleotide duplex and the second nick site being located within the second polynucleotide strand of polynucleotide duplex. A spacer region separates the first and second nick sites from each other, thereby linearly offsetting the first nick site from the second nick site, i.e., there is a linear offset between the first nick site and the second nick site. Further, each terminal end of the EA can be configured for ligation to both ends of the target DNA template. One or both of the nick sites, for example, facilitate polymerase binding and extension.

In certain example aspects, the linear end adapter includes a first Y-branch element sequence attached to the 5′ end flanking the first nick site and/or a second Y-branch element sequence attached to the 5′ end flanking the second nick site. The Y-branch element, for example, can encode a primer binding sequence or other beneficial sequence.

In certain example aspects, the first polynucleotide strand and/or the second polynucleotide strand of the EA includes a unique molecular identifier (UMI) sequence. For example, the UMI can be located within the spacer region. In certain examples aspects, the first polynucleotide strand of the EA includes a first sequence index (SID) and/or the second polynucleotide strand of the EA includes a second SID.

In certain example aspects, provided is a method of preparing a double-length DNA template a from target DNA template. The method includes, for example, performing a ligation reaction between a target DNA template and the end adapter as described herein to form a circular construct. For example, the target DNA template includes a first target DNA template terminal end and a second target DNA template terminal end. The ligation reaction thus (i) joins the first terminal end of the end adapter to the first target DNA template terminal end and (ii) joins the second terminal end of the end adapter to the second target DNA template terminal end. This forms the circular construct. Thereafter, a DNA polymerase-mediated extension reaction is performed on the circular construct. For example, the circular construct is contacted with multiple strand-displacement polymerases to initiate the extension reaction. The extension reaction forms a double-length DNA template, which includes, for example, a first copy and a second copy of the target DNA template.

In certain example aspects, the first copy of the target DNA template and the second copy of the target DNA template—of the double-length DNA template—are contiguously joined to each other by a DNA bridge region. The bridge region, for example, is derived from the end adapter. The bridge region, for example, is double-stranded.

In certain examples aspects, each polynucleotide strand of the double-length DNA template includes a 5′ to 3′ parental strand of the target DNA template and a 5′ to 3′ daughter strand copy of the parental strand of the target DNA template. In certain examples aspects, the parental strand of the target DNA template and the daughter strand copy of the target DNA template can be contiguously joined to each other by a 5′ to 3′ strand of the DNA bridge region.

In certain example aspects, the strand of the bridge region includes a unique molecular identifier (UMI) or a sequence index (SID). For example, the double-length DNA template includes a first terminal end and a second terminal end, with the first terminal end and/or the second terminal end including an SID.

In certain example aspects, such as when the linear end adapter includes a first Y-branch element sequence and a second Y-branch element sequence, the DNA polymerase-mediated extension reaction positions the first Y-branch element sequence and the second Y-branch sequence at the 5′ end of each parental strand of the double-length DNA template. Further, the polymerase-mediated extension reaction of the DNA circular construct synthesizes a first daughter Y-branch element sequence and a second daughter Y-branch element sequence, with the first daughter Y-branch element sequence being complementary to the first Y-branch element sequence and the second daughter Y-branch element sequence being complementary to the Y-branch element sequence. The Y-branch element, for example, can encode a primer binding site for subsequent PCR reactions.

In certain example aspects, the methods can be serially repeated. For example, serially repeating the method can produce a quadruple-length DNA template or a multi-length DNA template. In such example aspects, the multi-length DNA template includes multiple copies of the target DNA template.

In certain example aspects, provided is a method of identifying epigenetic information associated with a target nucleic acid sequence. The method includes, for example, ligating a linear target DNA template to both ends of the linear end adapter as described herein, thereby forming a circular DNA construct. A DNA polymerase-mediated bidirectional extension reaction is then performed on the circular DNA construct, in the presence of a plurality of protected cytosine nucleotides. A double-length DNA template is then formed, which includes the protected cytosine nucleotides, for example, in the newly synthesized strands. The double-length DNA template is then denatured and subjected to a bisulfite conversion reaction, which forms bisulfite-converted double-length DNA template strands of the double-length DNA template. A polymerase chain reaction (PCR) amplification reaction is then performed using the bisulfite-converted double-length DNA template strands, followed by a sequencing reaction of the PCR-amplified/bisulfite-converted double-length DNA template strands. Based on the sequencing of the PCR-amplified/bisulfite-converted double-length DNA template strands, epigenetic information associated with a target nucleic acid is identified. That is, bioinformatics analysis can be used to identify the epigenetic information.

In certain example aspects, each polynucleotide strand of the double-length DNA template of the method of identifying epigenetic information includes a parental template strand from the target DNA template and a daughter copy strand of the parental template strand. The parental template strand, for example, is contiguously joined to the daughter copy strand of the parental template strand by a single-stranded bridge region (with the single-stranded bridge region being derived from the end adapter). Further, during the DNA polymerase-mediated bidirectional extension reaction, the protected cytosine nucleotides are incorporated into the daughter copy strand of the parental template strand.

In certain example embodiments, sequencing of the PCR-amplified bisulfite-converted double-length DNA template strands provides a polynucleotide sequence for the parental template strand and a sequence for the daughter copy strand. The step of identifying the epigenetic information associated with the target nucleic acid then includes an intra-strand comparison of the polynucleotide sequence of the parental template strand with the polynucleotide sequence of the daughter copy strand. For example, a sequence discrepancy location between the polynucleotide sequence of the parental template strand and the polynucleotide sequence of the daughter copy strand identifies an unprotected cytosine residue location in the parental template strand. The unprotected cytosine residue location in the parental template strand, for example, corresponds to an unprotected cytosine residue location in the target nucleic acid sequence.

In certain example aspects, the double-length DNA template of the method of identifying epigenetic information includes a first copy and a second copy of the target DNA template. The first copy and the second copy of the target DNA template, for example, can be joined together by a double-stranded bridge region, with the bridge regions being derived from the end adapter. Further, each copy of the target DNA template within the double-length DNA template includes a parental template strand and a daughter strand that is complementary and hybridized to the parental template strand. During the DNA polymerase-mediated bidirectional extension reaction, for example, the protected cytosine nucleotides are incorporated into the hybridized complementary daughter strand.

In such example aspects, when the PCR-amplified bisulfite-converted double-length DNA template is sequenced, inter-strand comparison of the polynucleotide sequence of the parental template strand with the polynucleotide sequence of the hybridized complementary daughter strand can be used to identify epigenetic information associated with the target nucleic acid. For example, a nucleotide mismatch location between the polynucleotide sequence of the parental template strand and the hybridized complementary daughter identifies an unprotected cytosine residue location in parental template strand, with the unprotected cytosine residue location in the parental template strand corresponding to an unprotected cytosine residue location in the target nucleic acid sequence.

In certain example aspects, the protected cytosine nucleotides include methylated cytosine residues. In certain example aspects, the unprotected cytosine nucleotides are unmethylated cytosine residues. In certain example aspects, the double-length DNA template of the method of identifying epigenetic information includes a unique molecular identifier (UMI) and/or one or more sequencing indexes (SIDs).

In certain example aspects, provided is a double-length DNA template formed by the methods and compositions described herein. For example, the double-length DNA template includes a first copy and a second copy of target DNA template, with the first copy and the second copy of the target DNA template being contiguously joined to each other by a double-stranded bridge region. Further, each polynucleotide strand of the double-length DNA template incudes includes a parental template strand from the target DNA template and a daughter strand copy of the parental template strand. The parental template strand is contiguously joined to the daughter copy strand of the parental template strand, for example, by a strand of the bridge region. Additionally, each copy of the target DNA template within the double-length DNA template includes a parental template strand and a daughter strand that is complementary and hybridized to the parental template strand.

In certain example aspects, the double-length DNA template includes a first terminal end and a second terminal end, where either terminal end includes a sequence encoding a primer binding site. In certain example aspects, the bridge region—or a strand thereof—includes a unique molecular identifier (UMI) and/or a sequencing index (SID).

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

FIG. 1A is an illustration of a linear end adapter for synthesizing a double-length DNA template, in accordance with certain example embodiments.

FIG. 1B is a schematic depicting circularization of a target DNA template using an EA, in accordance with certain example embodiments.

FIG. 1C is a schematic depicting polymerase attachment and initiation of bidirectional extension of a circular construct, in accordance with certain example embodiments.

FIG. 1D is a schematic depicting continued polymerase extension of the circular construct and formation of the double-length DNA template, in accordance with certain example embodiments.

FIG. 2A is an illustration showing a Y-branched end adapter 200 (YBEA), in accordance with certain example embodiments.

FIG. 2B is a schematic depicting circularization of a target DNA template using the YBEA 200, in accordance with certain example embodiments.

FIG. 2C is a schematic depicting polymerase attachment and initiation of bidirectional extension of a circular construct including the YBEA 200, in accordance with certain example embodiments.

FIG. 2D is a schematic depicting continued polymerase extension of the circular construct and formation of the target DNA template using the YBEA 200, in accordance with certain example embodiments.

FIG. 2E is an illustration showing the double-length DNA template of FIG. 2D (lower panel) in a denatured (single-stranded) form, in which the original Y-branch elements provide a predetermined oligonucleotide primer binding sequence when replicated.

FIG. 3A is an illustration showing a Y-Branch End Adapter that includes a UMI (“YB-UMI-EA”), in accordance with certain example embodiments.

FIG. 3B is a schematic depicting circularization of a target DNA template using the YB-UMI-EA 300, in accordance with certain example embodiments.

FIG. 3C is an enlarged view of a portion of the target DNA template of FIG. 3B, showing an example nucleic acid sequence, in accordance with certain example embodiments.

FIG. 3D is a schematic depicting polymerase attachment and initiation of bidirectional extension of a circular construct including the YB-UMI-EA 300, in accordance with certain example embodiments.

FIG. 3E is a schematic depicting continued polymerase extension of the circularized target DNA template and formation of the double-length DNA template using the YB-UMI-EA 300 example embodiment, in accordance with certain example embodiments.

FIG. 3F is a schematic showing an example bisulfite conversion of the double-length DNA template and its PCR-amplified products, via the use of the Y-branch end adapter with a UMI (i.e., YB-UMI-EA) of FIG. 3A, in accordance with certain example embodiments.

FIG. 3G is a schematic showing both intra-strand and inter-strand bioinformatic analyses of a portion of the double-length DNA template to ascertain epigenetic information associated with the original target DNA template, in accordance with certain example embodiments.

FIG. 4A is an illustration showing a Y-branch end adapter that includes two SID sequences and a UMI (i.e., a YB-UMI/SID-EA), in accordance with certain example embodiments.

FIG. 4B is an illustration showing a double-length DNA template that arises from use of the YB-UMI/SID-EA 400 of FIG. 4A, in accordance with certain example embodiments.

FIG. 5A is an illustration showing a modified Y-branched end adapter according to FIG. 2A, but that has been modified so that it accommodates only a single polymerase attachment and unidirectional extension, in accordance with certain example embodiments.

FIG. 5B is a schematic depicting polymerase attachment and initiation of unidirectional extension of a circular construct, in accordance with certain example embodiments.

FIG. 5C is a schematic depicting continued polymerase extension of the circular construct and formation of the asymmetric template using the modified YBEA 500, in accordance with certain example embodiments.

FIG. 6 is a schematic depicting the formation of a quadruple-length DNA template from a double-length DNA template, in accordance with certain example embodiments.

### DETAILED DESCRIPTION OF THE INVENTION

### Overview

Disclosed herein are DNA library preparation methods and compositions that duplicate a target nucleic acid sequence. For example, a target DNA template including or encoding the target nucleic acid sequence is extended by adding a single copy of the target DNA template to the original target DNA template, thereby forming a double-length DNA template. That is, the double-length DNA template is “double length” in that it includes two copies of the original, target DNA template (and hence two copies if the target sequence). Generally, the methods include, for example, the steps of circularizing the target DNA template followed by replication to form two copies of the target DNA template, each copy located within the double-length DNA template.

Beneficially, each strand of the double-length DNA template includes a parental polynucleotide sequence contiguously joined to a newly synthesized daughter copy of the parental polynucleotide sequence. Further, each copy of the target DNA template within the double-length DNA template includes a parental strand hybridized to a complementary daughter DNA strand. In certain examples, predetermined sequences can also be included in the double-length DNA template, such a primer sequences, unique molecule identifiers (UMIs), sample indexes (SIDs), and the like. And with the association of parental and daughter polynucleotide sequences within the double-length DNA template, sequencing of the double-length DNA template can beneficially reveal genetic and epigenetic information associated with the target nucleic acid sequence.

To facilitate the preparation of the double-length DNA template, in certain examples provided is a linear end adapter (EA) that includes hybridized polynucleotide strands, thus forming a polynucleotide duplex, such as a DNA molecule. For example, the ends of an EA are each ligated to opposing ends of a target DNA template to form a circular construct. The EA includes juxtaposed nick sites—one on each polynucleotide strand—that are separated by a spacer region. Because each nick site resides in the polynucleotide strands of the EA duplex, each nick site is flanked by a 5′ end and a 3′ end. As such, in certain examples the EA provides an exposed 3′ end for polymerase binding and extension on each strand of the EA.

For example, when a circular construct including the EA is contacted with DNA polymerases, the two juxtaposed 3′ ends can be extended by a polymerase in opposite directions, while the opposing strands of the target DNA template are displaced. Complete extension of both free 3′ ends provided by the EA yields a double-length DNA template, with each copy of the target DNA template within the double-length DNA template including one original (parental) DNA strand and one newly synthesized and complementary daughter strand. Each copy of the target DNA template is separated by the EA, the EA forming a bridge between the two template copies. In this way, the bridge of the double-length DNA template is derived from the EA. Further, each polynucleotide strand of the double-length DNA template includes a parental polynucleotide sequence from the target DNA template and a new daughter copy of the parental polynucleotide sequence, the parental sequence and daughter copy being contiguously and covalently joined to each other and having the same sequence.

In certain examples, single-stranded (ss) branching sequence elements (or Y-branch elements) can be added to the 5′ end of each nick site of the EA, forming one or more Y-branch end adapters within the double-length DNA template. The Y-branch elements can include, for example, a polynucleotide sequence that encodes a primer binding site. For example, the Y-branch elements can include a single-stranded polynucleotide sequence (e.g., ssDNA), the complement of which encodes a primer binding site as described herein. The primer binding sites can be used, for example, in a subsequent PCR reaction to efficiently and accurately amplify the double-length DNA template (thereby amplifying the original target DNA template).

In certain examples, because the methods disclosed herein advantageously provide a double-length DNA template in which a parent polynucleotide sequence is covalently and contiguously linked to a daughter polynucleotide strand copy, both epigenetic (parent strand) and genetic (daughter strand) information are preserved in the double-length DNA template. That is, because both polynucleotide strands of the double-length DNA template compositions provided herein include a parental polynucleotide sequence from the target DNA template and a daughter copy of that parental sequence, strand-specific analysis and comparison can be used to identify parental strand methylation, thereby discerning epigenetic information associated with the parental strand and hence as present in the target sequence. Further, such genetic and epigenetic information can beneficially be obtained in a single read by sequencing the double-length DNA template.

In certain examples, the methods provided herein can be used to create a double-length DNA template that includes a Unique Molecule Identifier (UMI). The UMI, for example, can be included in the spacer region of the end adapter provided herein, i.e., in the region between the juxtaposed nick sites of the end adapter. In such examples, the Y-branch elements can also be included to allow for subsequent PCR amplification. By including UMIs in the double-length DNA template, for example, the double-length DNA template can be used in a variety of bioinformatic applications. For example, sequence information from each strand of the double-length DNA template can be bioinformatically paired to advantageously confirm the accuracy of the sequence reads. Such UMIs can also, in certain examples, aid in strand differentiation for the genetic and epigenetic analyses described herein.

In certain examples, the methods provided herein can beneficially be used to create double-length DNA template compositions that include one or more sample indexes (SIDs). Conventionally, use of such SIDs are highly useful in applications such as DNA multiplexing, i.e., the processing of multiple, different samples at the same time. For example, different SIDs can be included adjacent to the Y-branch sequence elements described herein. Thereafter, double-length DNA molecules with different SIDs can be processed simultaneously, the SIDs allowing differentiation of the samples following sequencing. Further, because multiple copies of an SID can appear in a single, duplicated PCR product strand, bioinformatically the SID may be determined with high accuracy, thereby reducing or eliminating the need for additional for error correction. In such example embodiments, the SIDs can also be used as landmarks in a given strand, allowing additional analytics.

In certain examples, the methods and compositions for producing the double-length DNA template can be applied serially to multiply the number of parental target DNA templates on the single molecule with each iteration, such as to create a quadruple length DNA template or multi-length DNA template. This can beneficially be used in sequencing applications, for example, to produce additional template reads on a single pass, thereby achieving higher read accuracy and confidence. In still other example examples, the target DNA template can be extended asymmetrically, resulting in an asymmetric DNA template. For example, a nick site of the end adapter can be blocked, thereby enabling extension from a single nick site.

Because the double-length DNA template can be limited to a single copy extension product (i.e., forming a double template of the original parental target DNA template), the methods and compositions provided herein beneficially also maintain library length uniformity and read efficiency. The methods and compositions provided herein also improve sequencing accuracy while balancing other important characteristics of a sequencing system such as throughput, efficiency, and read length. These and other examples and benefits will become apparent to the skilled artisan in view of the further detailed description provided herein.

### Terms & Nomenclature

The invention will now be described in detail by way of reference only using the following definitions and examples. All patents and publications, including all sequences disclosed within such patents and publications, referred to herein are expressly incorporated by reference in their entirety.

Unless defined otherwise herein, all technical and scientific terms used herein have the same meaning as commonly understood by one of ordinary skill in the art to which this invention belongs. Such common techniques and methodologies are described, for example, in Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library, each of which provide one of skill with a general dictionary of many of the terms used in this invention.

Although any methods and materials similar or equivalent to those described herein can be used in the practice or testing of the present invention, the preferred methods and materials are described. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

In addition, features, operations, or characteristics described in the specification can be combined in any appropriate manner to form various implementations of the example embodiments. Meanwhile, those skilled in the art will fully appreciate that certain steps or actions for describing a method can also be exchanged or adjusted in terms of order. Therefore, the various orders in the specification and the drawings are only for the purpose of clearly describing a certain embodiment, but are not the necessary orders, unless it is otherwise stated that a certain order must be followed or such an order is necessary from context (e.g., a polymerase must be added to a reaction mixture for polymerase-mediated replication to occur).

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

As used herein, the singular forms “a,” “an” and “the” include plural referents unless the context clearly dictates otherwise.

Ranges can be expressed herein as from “about” or “approximately” one particular value, and/or to “about” or “approximately” to another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect.

In certain example embodiments, the term “about” or “approximately” is understood as within a range of normal tolerance in the art, for example within 2 standard deviations of the mean. About or approximately can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary,” or “exemplified,” are not meant to show preference, but rather to explain that the aspect discussed thereafter is merely one example of the aspect presented.

The term “amplification” refers to a process of making additional copies of a target nucleic acid. Amplification can have more than one cycle, e.g., multiple cycles of exponential amplification. Amplification may have only one cycle (making a single copy of the target nucleic acid). The copy may have additional sequences, e.g., those present in the primers used for amplification. Amplification may also produce copies of only one strand (linear amplification) or preferentially one strand (asymmetric PCR).

As used herein, a “polymerase” refers to an enzyme that catalyzes the polymerization of nucleotide (i.e., the polymerase activity). Generally, the enzyme will initiate synthesis at 3′-end of the primer annealed to a polynucleotide template sequence and will proceed toward 5′ end of the template strand. A “DNA polymerase” catalyzes the polymerization of deoxynucleotides using a complementary template DNA strand and a primer, for example, by successively adding nucleotide to a free 3′-hydroxyl group. The template strand determines the sequence of the added nucleotide by Watson-Crick base pairing.

Generally, any DNA polymerase suitable for use with a rolling circle amplification reaction, for example, can be used in the replication reaction. In certain embodiments, a suitable DNA polymerase will possess strand displacement activity. The term strand displacement describes the ability to displace downstream DNA encountered during DNA synthesis. Several DNA polymerases with varying degrees of strand displacement activity are known in the art and commercially available. In certain example embodiments, the polymerase is a phi29 polymerase, bst polymerase, etc. In a preferred embodiment, the strand displacing polymerase is phi 29 polymerase.

In some embodiments, the DNA polymerase is a high-fidelity DNA polymerase. The fidelity of a DNA polymerase is the result of accurate replication of a desired template. Specifically, this involves multiple steps, including the ability to read a template strand, select the appropriate nucleoside triphosphate and insert the correct nucleotide at 3′ primer terminus, such that Watson-Crick base pairing is maintained. In addition to effective discrimination of correct versus incorrect nucleotide incorporation, some DNA polymerases possess a 3′→5′ exonuclease activity. This activity, known as “proofreading”, is used to excise incorrectly incorporated mononucleotides that are then replaced with the correct nucleotide.

In certain embodiments, suitable high-fidelity DNA polymerases for the practice of the present invention include KAPA HiFi DNA Polymerase, commercially available from Roche Diagnostics Corp., Q5® High-Fidelity DNA Polymerase, commercially available from New England Biolabs, Inc., and an engineered Pfu DNA polymerase, such as Pfu-X, commercially available from Jena Biosciences.

As used herein, the terms “ligate,” “ligating,” “ligation” and the like refer generally to the process for covalently linking two or more molecules together, for example, covalently linking two or more nucleic acid molecules to each other. The similar term “ligatable” refers to having the ability to ligate. As those skilled in the art will appreciate, ligation includes a condensation reaction that forms a covalent bond between an end of a first and an end of a second nucleic acid molecule.

In certain example embodiments, the ligation can include forming a covalent bond between a 5′ phosphate group of one nucleic acid and a 3′ hydroxyl group of a second nucleic acid thereby forming a ligated nucleic acid molecule. Generally, for the purposes of this disclosure, a target DNA template sequence can be ligated to an end adapter to generate a circularized construct. Ligation includes the joining of two DNA molecules that each have overhanging ends (i.e., “sticky” ends), that is one strand is longer than the other (typically by at least a few nucleotides), such that the longer strand has bases which are left unpaired. Ligation also includes the joining of DNA molecules where the strands of each molecule are equal length (i.e., “blunt ends” with no overhang).

In certain example embodiments, ligation can be achieved with asymmetric 5′ thymine base nucleotide overhangs on the target DNA template and 5′ adenine base nucleotide overhangs on the end adapter. For example, target DNA template and end adapters can be combined under equimolar or near-equimolar concentrations to perform the ligation. In certain example embodiments, concentrations of end adapter and target DNA template can be optimized through trial and error to favor the circularization ligation over concatenation, for example, molar ratios of target DNA template to adapter can be 1:1, 1:5, 1:10, 1:25, or 1:50. In certain example embodiments, improved circularization can be achieved when the target DNA template and/or end adapter includes sufficient flexibility to bend around and align for a sufficient time and frequency. It has been shown that ds-DNA>200 base pairs will ligate to form “minicircles” and that those with linear ds-DNA oligos with nick sites will circularize even more readily (see, e.g., “Small DNA Circles as Probes of DNA Topology”, Bates, A. D. et al., Biochem. Soc. Trans. (2013) 41, 565-570, which is incorporated by reference herein in its entirety). Sequencing libraries of interest are often in this size range. In certain example embodiments, the target DNA template is from 200 to 500 base pairs in length.

In certain example embodiments, circularization of the target DNA template can be facilitated by reducing the concentration of the target DNA template and/or end adapter to favor circularization over concatemerization. In other embodiments, circularization can be promoted through a “protein scaffolding” strategy that uses one or more DNA binding proteins to increase local concentration of intramolecular ligate-able ends to push equilibrium towards circularization and physically bend DNA to overcome energetic challenge of forming small circles. In certain example embodiments suitable DNA binding proteins for protein scaffolding include histones, Abf2p, DSP1, histone-like protein AU, and CAP. In certain example embodiments, circularized ligation constructs can be enriched for by treatment with one or more exonucleases, as the circularized constructs do not present free ends to initiate exonuclease-mediated DNA degradation. Certain exemplary exonucleases in ExoVIII, ExoIII, and T5 exonuclease.

As used herein, the terms “target” “target sequence” or “target nucleic acid sequence” are used interchangeably to refer to any nucleic acid molecule of interest that is subjected to processing, e.g., for generating a double-length DNA template as described herein. The target nucleic acid sequence can include or consist of genomic DNA, subgenomic DNA, chromosomal DNA (e.g., from an isolated chromosome or a portion of a chromosome, e.g., from one or more genes or loci from a chromosome), mitochondrial DNA, chloroplast DNA, plasmid or other episomal-derived DNA (or recombinant DNA contained therein), or double-stranded cDNA made by reverse transcription of RNA, or RNA that can be subsequently converted to cDNA through any art-recognized method. Further, the target nucleic acid sequence, such as target DNA or RNA, can be derived from any in vivo or in vitro source, including from one or multiple cells, tissues, organs, body fluids, or organisms, whether living or dead, or from any biological or environmental source (e.g., water, air, soil).

The terms “DNA,” “double-stranded DNA,” or “dsDNA” refer generally to complementary deoxyribonucleic acid polynucleotide strands that are hybridized to form a duplex. The two polynucleotide strands are held together by hydrogen bonds between the complementary nucleotide base pairs (i.e., Watson-Crick). Each nucleotide in DNA consists of a sugar molecule, a phosphate group, and one of four nitrogenous bases: adenine (A), cytosine (C), guanine (G), or thymine (T). The strands need not be perfectly complementary to maintain the duplex. Double-stranded DNA can be found in the nucleus of eukaryotic cells, as well as in the cytoplasm and plasmids of prokaryotic cells. It can also be used in various molecular biology techniques, such as PCR (polymerase chain reaction), DNA sequencing, and genetic engineering.

A DNA strand or single-stranded DNA (ssDNA) refers to one of the polynucleotide chains of the DNA molecule, which may also be referred to as ssDNA. A daughter polynucleotide strand, for example, is a new strand of the DNA duplex that is created from replicating a DNA molecule. For example, a polymerase-mediated replication reaction will use a template DNA strand to create complementary strand that is the daughter strand. In certain example embodiments, the DNA is cDNA that has been converted or otherwise derived from a target RNA sequence.

As used herein, the term “target DNA template” and “DNA template” are used interchangeably and refer to a DNA molecule that encodes or includes the genetic and/or epigenetic information of a target nucleic acid sequence. For example, one of the strands includes or encodes the target sequence, with the other hybridized and opposing strand of the DNA molecule being complementary to the strand including or encoding the target sequence. In certain embodiments, the target DNA template may be a natural DNA target fragment (e.g., a genomic or cell-free DNA target fragment) or it may be a cDNA copy of a natural DNA or RNA target fragment. The target DNA templates disclosed herein are the molecules that are replicated (e.g., duplicated) and/or subjected to DNA sequencing. Further, when a subsequent DNA molecule is formed including, for example, a polynucleotide strand of the target DNA template, the strand may be referred to as the “original” or “parental” strand of the target DNA template, indicating that the strand was originally part of the target DNA template. The target template, for example, can be made according to any means known in the art.

The term “primer” refers to a single-stranded oligonucleotide which hybridizes with a target nucleic acid sequence (“primer binding site”) and is capable of acting as a point of initiation of synthesis along a complementary strand of nucleic acid under conditions suitable for such synthesis. That is, the “primer” functions as a substrate on which nucleotides can be polymerized by a polymerase. In various embodiments, the primer has a free 3′-OH group that can be extended by a nucleic acid polymerase. For a template-dependent polymerase, typically at least the 3′ portion of the primer oligonucleotide is complementary to a portion of the template nucleic acid to which it “binds” (or “complexes,” “anneals,” or “hybridizes”) by hydrogen bonding and other molecular forces to the template to give a primer/template complex for initiating synthesis by the DNA polymerase, and is extended (i.e., “primer extension”) during DNA synthesis by the addition of covalently bound bases complementary to the template that are attached at their 3′ ends.

As used herein, Unique molecular identifiers (UMIs) are sequences of nucleotides inserted within or identified in DNA molecules that may be used to distinguish individual DNA molecules from one another. Due to their complementary nature in a DNA molecule, a UMI that is present or inserted into a DNA molecule can also be used to identify individual strands of a DNA molecule, inasmuch as the polarity (direction) of the UMI sequence can be identified and distinguished between two complement DNA strands. See, e.g., Kivioja, Nature Methods 9, 72-74 (2012). UMIs may be sequenced along with the DNA molecules with which they are associated to determine whether the read sequences are those of one source DNA molecule or another. The term “UMI” is used herein to refer to both the sequence information of a polynucleotide and the physical polynucleotide per se. UMI sequences may be random, pseudo-random or partially random, or nonrandom nucleotide sequences that are inserted within or otherwise incorporated into, for example, the end adapters as described herein.

The term “sample index” is a sequence of nucleotides that is appended to a target polynucleotide, where the sequence identifies the source of the target polynucleotide (i.e., the sample from which sample the target polynucleotide is derived). As such, a sample index (or SID) is also referred to as “sample identifier sequence,” “index sequence identifier,” “multiplex identifier” or “MID.” In use, each sample includes a different sample index sequence (e.g., one sequence is appended to each sample, where the different samples are appended to different sequences), and the samples are pooled. After the pooled sample is sequenced, the sample identifier sequence can be used to identify the source of the sequences. Conventionally, a sample identifier sequence may be added to the 5′ end of a polynucleotide or 3′ end of a polynucleotide. In certain cases, some of the sample identifier sequence may be at the 5′ end of a polynucleotide and the remainder of the sample identifier sequence may be at 3′ end of the polynucleotide. When elements of the sample identifier have sequence at each end, together, 3′ and 5′ sample identifier sequences identify the sample. In certain examples, the sample identifier sequence is only a subset of the bases which are appended to a target oligonucleotide. And as described herein, end adapters can be used to include a SID in to a sample.

As used herein, the term “polymerase chain reaction” (or “PCR”) refers to methods for increasing the concentration of a segment of a polynucleotide of interest in a mixture of genomic DNA without cloning or purification. See generally U.S. Pat. Nos. 4,683,195 and 4,683,202 (describing the PCR process). The process for amplifying a polynucleotide of interest consists generally of repeated cycles of denaturation, primer-annealing, and extension using a DNA polymerase enzyme. Since the amplified segments of the desired polynucleotides of interest become the predominant nucleic acid sequence (in terms of concentration) in the mixture, they are said to be “PCR amplified.” In a modification of the methods discussed above, the target nucleic acid molecules can be PCR amplified using a plurality of different primer pairs (in some cases, one or more primer pairs for each target nucleic acid molecule of interest) to form a multiplex PCR reaction.

As used herein, the term “end adapter” refers generally to a polynucleotide duplex, e.g., a DNA molecule, that can be added (i.e., joined to) to a target DNA template. An end adapter may be from 5 to 100 bases in length, and may provide, include, or code for an amplification primer binding site, a sequencing primer binding site, a molecular identifier and/or a sample identifier sequence, as described herein. The end adapter can be added to both 5′ end and 3′ end of a target DNA template via ligation. When added to a target DNA template, for example, end adapter forms a circularized structure (a “circularized DNA construct” or “circular construct”) in which both ends of the target molecule bind to the ends of the end adapter.

### Double-Length DNA Templates

Turning now to the drawings, in which like numerals indicate like (but not necessarily identical) elements throughout the figures, example embodiments are described in detail. Further, while certain of the figures provided herein illustrate target DNA template ligation, circularization, and replication of a single target DNA template, it is to be understood that that multiple target DNA templates are generally ligated, circularized, and replicated in a single library preparation reaction, such as when multiple reaction components are combined (e.g., multiple target DNA templates, end adapters, polymerase, etc.). The multiple replicates can then be used in any number of different applications, for example, such as sequencing or other analysis.

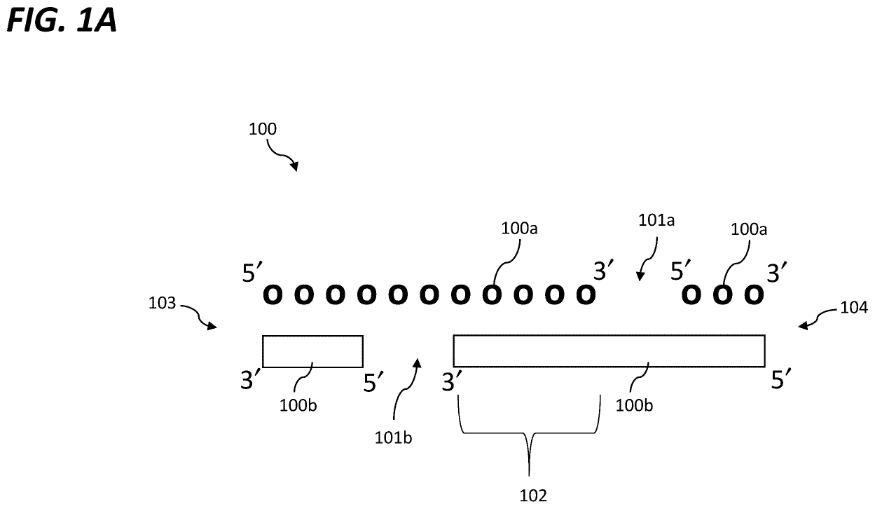

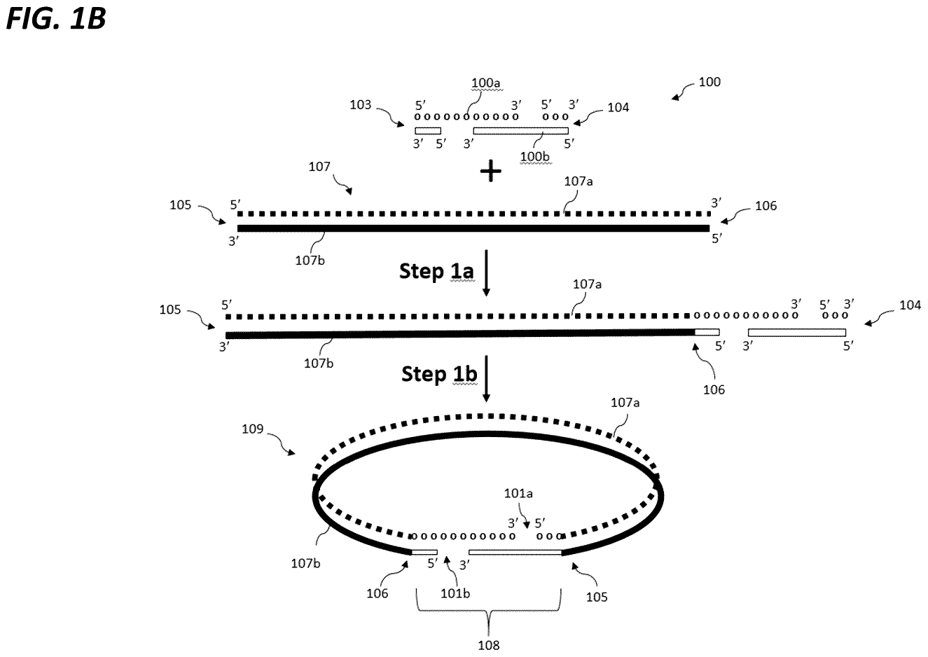

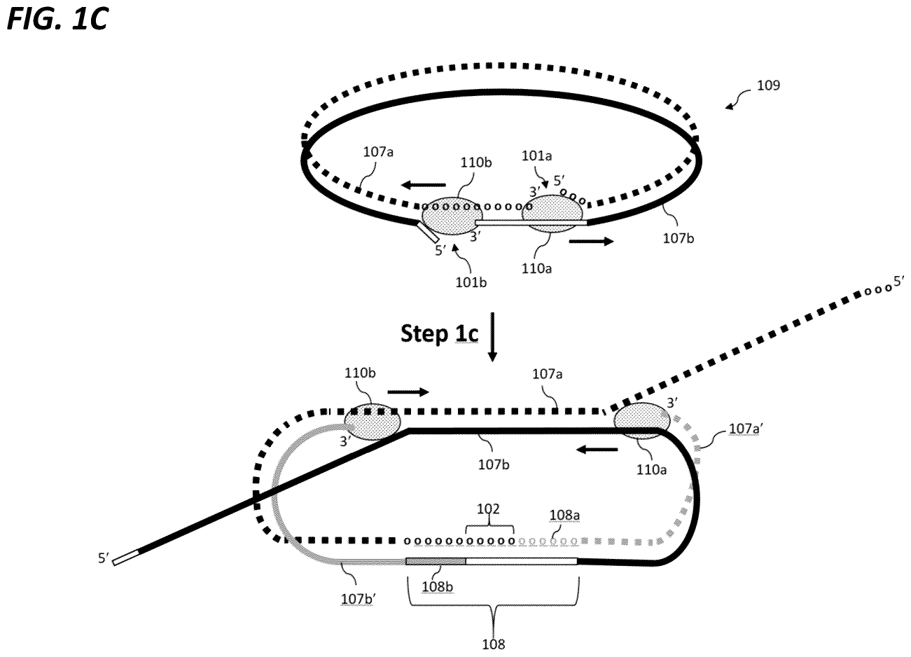

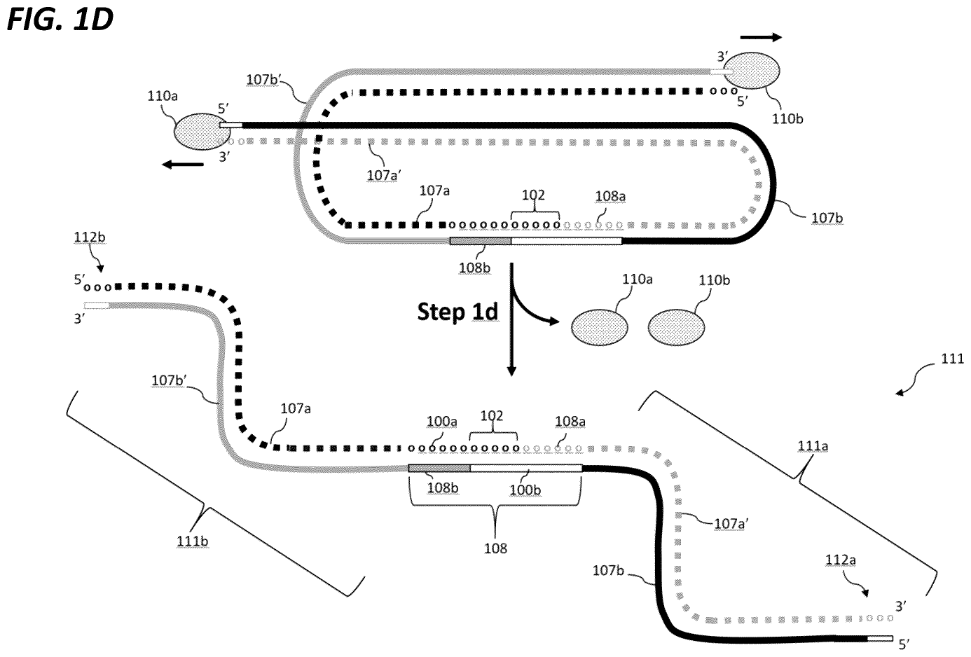

In certain example embodiments, provided is a method for preparing a DNA library, the method including synthesizing a double-length DNA template from a target nucleic acid via the use of a liner end adapter (EA). This is shown in FIGS. 1A-1D, which collectively illustrate the features of an example EA and show how the example EA can be used to synthesize the double-length DNA template, in accordance with certain example embodiments.

With reference to FIG. 1A, shown is a linear end adapter (EA) for synthesizing a double-length DNA template, in accordance with certain example embodiments. As shown, the EA 100 is a duplexed polynucleotide molecule, such as a DNA molecule, that includes hybridized oligonucleotide strands, i.e., first polynucleotide strand 100a (shown in circles) and hybridized second polynucleotide strand 100b (shown in rectangles). As used herein, in connection with the structures of the EAs of the present invention, the term “polynucleotide strand” refers to one or more oligonucleotides with the same 5′ to 3′ polarity that hybridize with a portion of one or more complementary oligonucleotides to form EA structure 100. In the embodiment depicted in FIG. 1A, polynucleotide strands 100a and 100b each include two oligonucleotide portions, separated respectively by nick site 101a and nick site 101b (as described further below). In this regard, the reference to “100a” refers to the entire 5′→3′ strand, with the nick site 101a within strand 100a. Likewise, the reference to “100b” refers to the entire 5′→3′ strand hybridized to strand 100a, with the nick site 101b within the strand 100b. In certain example embodiments, the entire length of the EA is from 50 to 100 nucleotides, such as 75-80 nucleotides in length. In certain example embodiments, the lengths of the oligonucleotides used to generate the EA are selected to ensure efficient and specific hybridization to form a stable EA structure, as discussed further herein.

As is also shown in the example EA of FIG. 1A, within the EA are a first nick site 101a and a second nick site 101b. That is, in certain example embodiments, the EA includes internal first and second nick sites 101a and 101b, one in each of the first and second polynucleotide strands 100a and 100b of the EA 100. The nick site, for example, includes any break or gap in one strand of the DNA molecule, such that the strand is not contiguous. In certain example embodiments, the nick site is a break or disruption of the phosphodiester backbone, while in other example embodiments the nick site is a gap of one or more nucleotides in the DNA strand. Notably, each nick site 101a and 101b is associated with—and flanked by—a free 5′ end and a free 3′ end. With reference to FIG. 1A, the depicted length and positions of the nick sites 101a and 101b is not intended to be limiting, but rather is shown for illustrative purposes only.

In certain example embodiments, by exposing a 3′ end in nick sites 101a and 101b, the EA can facilitate a polymerase-mediated strand extension reaction. That is, a polymerase can use the exposed 3′ end to extend 3′-associated strand in a conventional polymerization and strand displacement reaction, as described herein. Preferably, the nick sites 101a and 101b are spaced apart by spacer region 102 such that the EA can accommodate attachment of two polymerases for bidirectional extension, as described herein. As shown, for example, the spacer region linearly offsets the first nick site 101a from the second nick site 101b. Hence, the nick sites 101a and 101b can be spaced far enough apart, as separated by the spacer region 102, such that binding of one polymerase does not sterically hinder and/or displace the binding of a second polymerase. The EA 100 also includes terminal ends 103 and 104 flanking each nick site, each end 103 and 104 being compatible with efficient ligation to the ends of a target DNA template. That is, ends of the EA are ligatable to a target DNA template.

Any means known in the art can be used to form or otherwise create the EA. For example, as depicted in FIG. 1A, in one embodiment the EA is formed by hybridization of four synthetic oligonucleotides that are not fully contiguous, thereby leaving spacer regions (also referred to herein as “gaps” or “nicks”) upon hybridization. Any other suitable method for generating a nick, gap, or other site for polymerase binding and initiation of DNA synthesis may be used. For example, an EA can be generated from contiguous oligonucleotide strands designed to include recognition sites for one or more nicking enzymes (i.e., nicking endonucleases) that are suitably placed. Nicking enzymes are known in the art and hydrolyze (cut) only one strand of the DNA duplex, to produce DNA molecules that are “nicked”, rather than cleaved. Treatment of the EA with the nicking enzyme(s) generates the free 3′ ends that provide polymerase initiation sites in each strand.

With reference to FIG. 1B, provided is a schematic depicting circularization of a target DNA template using the EA 100, in accordance with certain example embodiments. The target DNA template, for example, includes or encodes the target sequence. Before ligation of adapters, the ends of the target DNA template can be prepared for ligation. For example, by end repair and creating blunt ends with 5′ phosphate groups. DNA templates may be rendered blunt-ended by a number of methods known to those skilled in the art. In a particular method, the ends of the fragmented DNA are “polished” with T4 DNA polymerase and Klenow polymerase, a procedure well known to skilled practitioners, and then phosphorylated with a polynucleotide kinase enzyme. A single ‘A’ deoxynucleotide is then added to both 3′ ends of the DNA molecules using Taq polymerase or Klenow exo minus polymerase enzyme, producing a one-base 3′ overhang that is complementary to the one-base 3′ ‘T’ overhang on the double-stranded end of an adaptor.

As shown in FIG. 1B, the double-stranded EA 100 is combined with a target DNA template 107, the target DNA template 107 having a first terminal end 105 and a second terminal end 106. As is also shown, the target DNA template includes complementary polynucleotide strands, i.e., a first template strand 107a (dashed line) and a second template strand 107b (solid line), both of which are referred to herein as the “parent strands” or “parental strands.” That is, the strands 107a and 107b of the target DNA template 107 correspond to the original strands of the target DNA template, the target DNA template including or encoding the target sequence as described herein. In some embodiments, the parent strands will include epigenetic information, e.g., methylated cytosine residues.

At Step 1a, for example, the EA 100 is ligated to either end of the target DNA template 107, the target DNA template including parental polynucleotide strands 107a and 107b. For example, terminal end 103 of the EA 100 is ligated to template end 106 (FIG. 1B). Alternatively at Step 1a, and although not shown for simplicity, the other terminal end of the EA 100 (i.e., 104) is ligated to terminal end 105 of the target DNA template 107. Either way, one end of the EA 100 is joined to the end of the target DNA template.

At Step 1b of FIG. 1B, the remaining free end of the EA 100 is ligated to the remaining free end of the target DNA template 107 to form a circular construct 109. For example, if terminal end 103 of the EA 100 is ligated to template end 106 at Step 1a, then at Step 1b EA terminal end 104 of EA 100 is ligated to template terminal end 105, thereby forming the circular construct 109 of the original (parental) template 107. Alternatively, if terminal end 104 of the EA 100 is ligated to template end 105 at Step 1a, then at Step 1b EA terminal end 103 is ligated to template terminal end 106, thereby forming the circular construct 109. Either way, at Step 1b of FIG. 1B the two terminal ends 103 and 104 of the EA 100 join each end 105 and 106 of the template 107, thereby forming a DNA bridge 108 between the ends of the template 107. That is, the entirety of the EA 100 forms the DNA bridge 108 between the two ends 105 and 106 of the parental template 107. This forms the circular construct 109 that includes the EA 100 (as bridge 108) along with complementary parental template strands 107a and 107b of the parental template 107.

In this way, the EA 100 (of FIG. 1A) operates as bridge precursor for the bridge region 108 of the circular construct 109. As shown, the respective first and second nick sites 101a and 101b remain in the circular construct 109 (as part of the bridge region 108) and hence, in certain example embodiments, provide two respective 3′ ends available for polymerase attachment and bidirectional extension, as described herein.

Continuing with the above example, FIG. 1C is a schematic depicting polymerase attachment and initiation of bidirectional extension of a circular construct 109, in accordance with certain example embodiments. As shown, once the circular construct 109 is formed (at Step 1b, FIG. 1B), DNA polymerases, shown as DNA polymerase 110a and 110b, are added to initiate a replication reaction. For example, in one embodiment, DNA polymerase 110a attaches to the nick site 101a of the EA 100. In this regard, the nick site 101a, with its available 3′ end, functions a primer end for DNA polymerase 101a attachment and extension initiation. Similarly, DNA polymerase 110b attaches to the nick site 101b of the EA 100, with the 3′ end nick site 101b functioning as a primer end for DNA polymerase 110b attachment and extension. As shown (with opposing arrows), polymerases 110a and 110b are positioned for bidirectional extension of the circular construct 109 in opposite directions (FIG. 1C, top panel).

At Step 1c of FIG. 1C, the first and second polymerases 110a and 110b bidirectionally extend the circular construct 109 in opposite directions (FIG. 1C, lower panel, see arrows). For example, polymerase 110a extends 3′ end of nick site 101a, while also displacing 5′ end of nick site 101a (and its associated parental template strand 107a). That is, as polymerase 110a proceeds, it extends the 3′ end of nick site 101a using parental strand 107b as a template to synthesize the new, daughter strand 107a′ that is complementary to the sequence of parental strand 107b (and that hence shares the same sequence of parental strand 107a). The new daughter strand 107a′ also includes, as part of the DNA bridge region 108, replicated ssDNA daughter strand bridge portion 108a.

Likewise, polymerase 110b extends 3′ end of nick site 101b, while also displacing 5′ end of nick site 101b (and its associated parental template strand 107b), using parental strand 107a as a template (FIG. 1C, lower panel). In other words, as polymerase 110b proceeds at Step 1c, it extends 3′ end of nick site 101b using parental strand 107a as a template to synthesize a new, daughter strand 107b′ that is complementary to the sequence of parental strand 107a (and that hence shares the same sequence of parental strand 107b). The new daughter strand 107b′ also includes, as part of the bridge region 108, replicated ssDNA daughter strand bridge portion 108b.

FIG. 1D is a schematic depicting continued polymerase extension of the circular construct 109 and formation of the double-length DNA template, in accordance with certain example embodiments. As shown (top panel), polymerases 110a and 110b continue to the end of the parental template 107. For example, polymerase 110a continues along parental template strand 107b to 5′ end of parental template strand 107b, completing the synthesis of new daughter strand 107a′. Likewise, polymerase 110b continues along parental template strand 107a to the 5′ end of parental template strand 107a, completing the synthesis of new daughter strand 107b′. At Step 1d, once polymerases 110a and 110b complete synthesis of daughter strands 107a′ and 107b′, respectively, the polymerases 110a and 110b dissociate from the circular construct 109, forming the double-length DNA template 111 (as shown in FIG. 1D, lower panel).

As depicted in FIG. 1D (lower panel), the double-length DNA double template 111 includes two copies of the original target DNA template 107, i.e., first and second copies 111a and 111b, respectively, each flanking the bridge region 108, the bridge region 108 including spacer region 102. Notably, each template copy includes both a parental polynucleotide strand (shown in black) and a newly synthesized daughter polynucleotide strand (shown in gray). For example, template copy 111a includes original (parental) template strand 107b and newly synthesized daughter strand 107a′. On the other side of the bridge region 108, template copy 111b includes original (parental) template strand 107a and newly synthesized daughter strand 107b′. Further, the double-length DNA 111 template includes a first terminal end 112a and a second terminal end 112b. The first terminal end 112a, for example, includes the portion of the EA 100 strand 100b associated with 5′ end of nick site 101b EA 100 (open black rectangle at terminal end 112a) and its copy (open gray circles at terminal 112a). Likewise, second terminal end 112b of the double-length DNA template 111 includes the portion of the EA 100 strand 100a associated with 5′ end of nick site 101a EA 100 (open black circles at terminal end 112b) and its copy (open gray rectangle at terminal end 112b).

Notably, both strands of the double-length DNA template also include a parental strand (in black) joined to a newly synthesized daughter copy (in gray) of the parental strand. For example, parental strand 107a is covalently and contiguously joined to newly synthesized daughter strand 107a′ via a strand of the bridge region 108 (i.e., the strand of the bridge region 108 including strand portions 100a and 108a) in a 5′→3′ direction. Further, because of the polymerase-mediated extension of the circular construct 109 as described herein, the nucleotide sequence of parental strand 107a matches that of new daughter stand 107a′. That is, daughter strand 107a′ is a sequence copy (i.e., a daughter copy) of the parental strand 107a of the target DNA template.

Likewise on the complementary strand of the double-length DNA template, parental strand 107b is covalently and contiguously joined to new daughter strand copy 107b′ via a strand of the DNA bridge region 108 (i.e., the strand of the bridge 108 including strand portions 100b and 108b), also in a 5′→3′ direction. And similarly—and again because of the polymerase-mediated extension of the circular construct as described herein—the nucleotide sequence of parental strand 107b matches that of new daughter stand 107b′. In this way, each strand of the double-length DNA includes both a parental polynucleotide sequence and a daughter polynucleotide sequence copy on each strand, in addition to the parental template strand and its complementary daughter strand on each of the two target DNA copies 111a and 111b (FIG. 1D, lower panel).

Double-Length DNA Templates with Y-Branched End Adapters

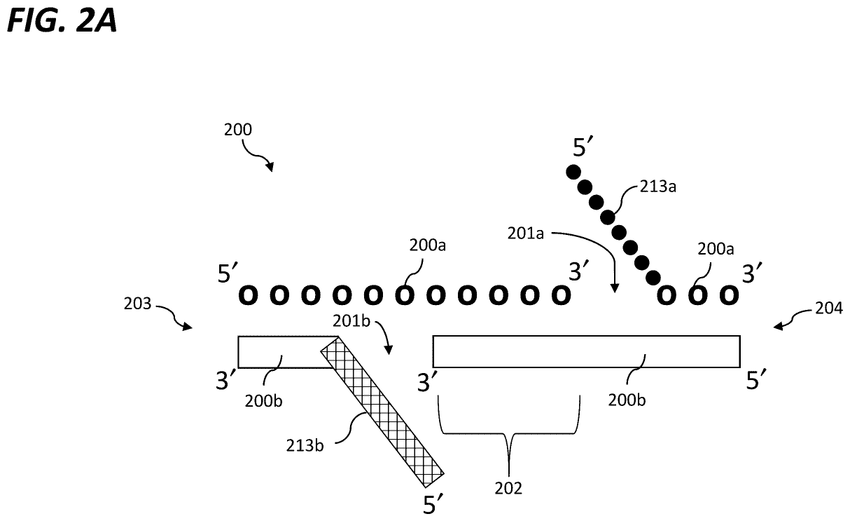

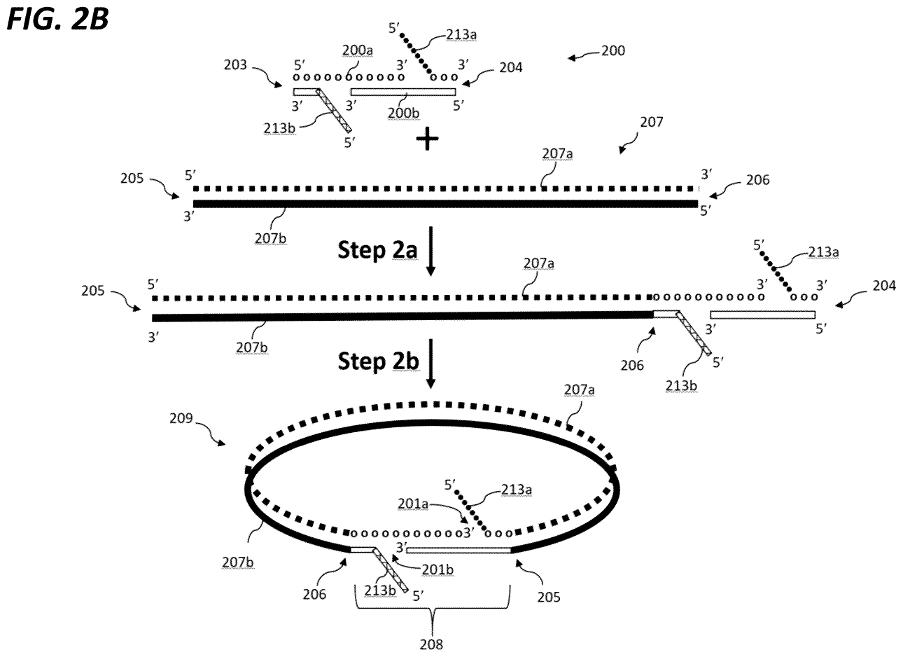

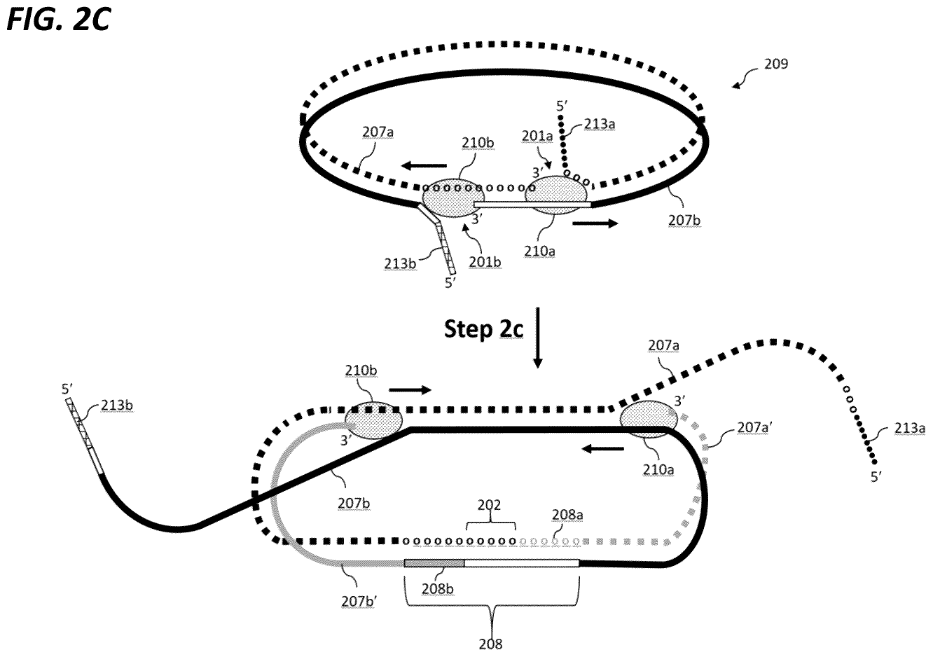

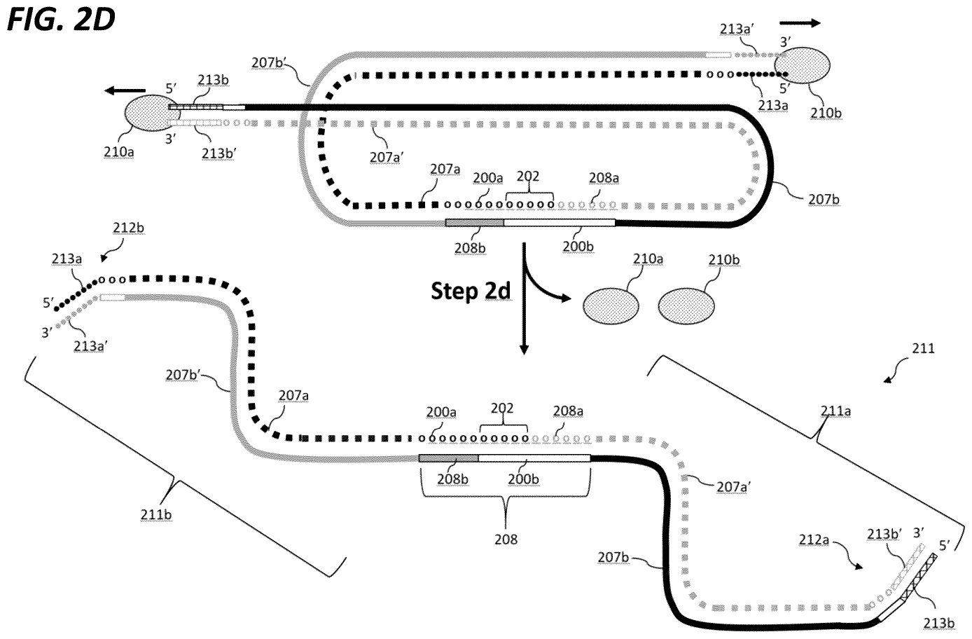

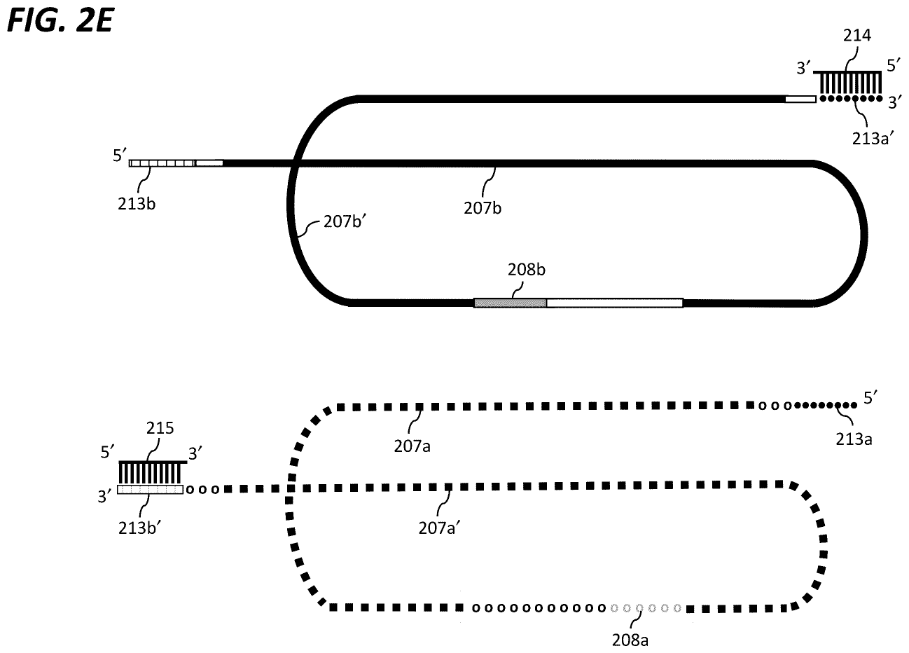

In certain example embodiments, the design of the end adapter (EA) as illustrated in FIG. 1A can be modified to confer additional features to the resultant double-length DNA template. This includes, for example, features that facilitate subsequent PCR amplification and/or DNA sequencing. This is shown in FIGS. 2A-2E, which collectively illustrate a modified EA and depict how the modified EA can be used to synthesize the double-length DNA template that includes primer binding sequences, in accordance with certain example embodiments.

With reference to FIG. 2A, provided is an illustration showing a Y-branched end adapter 200 (YBEA) having hybridized strands 200a (circles) and 200b (rectangles), in accordance with certain example embodiments. As shown, the YBEA 200 has the general duplexed polynucleotide EA structure as in FIG. 1A, except that a first Y-branch element 213a and second Y-branch element 213b is joined to the 5′ end of the first and second nick sites 201a and 201b, respectively. Each Y-branch element 213a and 213b, for example, can include a predetermined oligonucleotide sequence, the design of which can be tailored to achieve a specific objective, such as, but not limited to, PCR amplification or DNA sequencing the double-length DNA template. Also like the EA of FIG. 1A, the reference to “200a” refers to the entire 5′→3′ strand of the YBEA 200, with the nick site 201a within strand 200a. Similarly, the reference to “200b” refers to the entire 5′→3′ strand hybridized to strand 200a, with the nick site 201b within the strand 200b.

In certain example embodiments, each Y-branch element 213a and 213b sequences can include a predetermined oligonucleotide sequence that provides a complementary or hybridizable primer binding site useful for, e.g., PCR amplification. That is, each Y-branch element 213a and 213b, for example, can include 10-30 nucleotides, such as 15-25 nucleotides or 18-22 nucleotides, the complement of which includes a primer binding site sequence. In certain example embodiments, the Y-branch element 213a and 213b include the same sequence, while in other example embodiments the Y-branch element 213a and 213b include different sequences. In certain example embodiments, the Y-branch element 213a and 213b are the same length, while in other example embodiments the Y-branch element 213a and 213b may be different lengths.

The YBEA 200 also includes terminal ends 203 and 204 flanking each nick site 201a and 201b, each end 203 and 204 being compatible with efficient ligation to the ends of a target DNA template. That is, the terminal ends are ligatable to a target DNA template. Preferably, the nick sites 201a and 201b are spaced apart by spacer region 202 such that the EA can accommodate attachment of two polymerases for bidirectional extension, as described herein. That is, the nick sites 201a and 201b are far enough apart such that binding of one polymerase does not sterically hinder and/or displace the binding of a second polymerase. This configuration is shown in FIG. 2A, for example, where the spacer region 202 linearly offsets the first nick site 201a from the second nick site 201b.

FIG. 2B is a schematic depicting circularization of a target DNA template using the YBEA 200, in accordance with certain example embodiments. With reference to FIG. 2B, the YBEA 200, and its respective first and second Y-branch elements 213a and 213b associated with ends 203 and 204, respectively, is combined with a target DNA template 207 to form a circular construct 209 (akin to formation of the circular construct 109 of FIG. 1B). That is, the YBEA 200 is combined with a target DNA template 207, the target DNA template 207 having first and second terminal ends 205 and 206 and including complementary strands 207a and 207b (FIG. 2B).

At Step 2a, for example, the YBEA 200 is ligated to either end of the target DNA template 207, the target DNA template 207 including parental polynucleotide strands 207a and 207b. For example, terminal end 203 of the YBEA 200 is ligated to template end 206. Alternatively at Step 2a, and though not shown for simplicity, the other terminal end of the YBEA 200 (i.e., 204) is ligated to terminal end 205 of the target DNA template 207.

At Step 2b of FIG. 2B, the un-ligated (free) end of the YBEA 200 is ligated to the remaining free end of the target DNA template 207 to form a circular construct 209. For example, if terminal end 203 of the YBEA 200 is ligated to template end 206 at Step 2a, then at Step 2b YBEA terminal end 204 of YBEA 200 is ligated to template terminal end 205, thereby forming a circular construct 209 of the original (parental) target DNA template 207. Alternatively, if terminal end 204 of the YBEA 200 is ligated to template end 205 at Step 2a, then at Step 2b YBEA terminal end 203 is ligated to template terminal end 206, thereby forming the circular construct 209.

Either way, at Step 2b of FIG. 2B, the two, terminal ends 203 and 204 of the YBEA 200 join each end 205 and 206 of the template 207, thereby forming an YBEA bridge region 208 between the ends of the template 207. That is, the entirety of the YBEA 200 forms the bridge region 208 between the two ends 205 and 206 of the parental target DNA template 207. This forms the circular construct 209 that includes the YBEA 200 (as bridge region 208) along with complementary parental template strands 207a and 207b of the parental target DNA template 207. In this way, the YBEA 200 (FIG. 2A) operates as bridge precursor for the bridge region 208. Further, the respective first and second nick sites 201a and 201b remain in the circular construct 209 (as part of the bridge region 208) and hence provide a two respective free 3′ ends available for polymerase attachment and bidirectional extension, as described herein. The Y-branch elements 213a and 213b are also present in the circular construct 209.

Continuing with the above example, FIG. 2C is a schematic depicting polymerase attachment and initiation of bidirectional extension of a circular construct including the YBEA 200, in accordance with certain example embodiments. As illustrated in FIG. 2C (and akin to FIG. 1C), when first and second polymerases 210a and 210b are combined with the circular construct 209, they bind to nick sites 201a and 201b, respectively, and proceed in opposite directions (as indicated by the arrows in FIG. 2C, top panel). The polymerases 210a and 210b also displace 5′ end of parental strands 207b and 207a, respectively (including their associated and respective 213b and 213a Y-branch elements).

At Step 2c (FIG. 2C, lower panel), polymerases 210a and 210b bidirectionally extend the circular construct 209 in opposite directions (see arrows). That is, as polymerase 210a proceeds, it extends 3′ end of nick site 201a using parental strand 207b as a template to synthesize the new, daughter strand 207a′ that is complementary to the sequence of parental strand 207b (and that hence shares the same sequence of parental strand 207a). The new daughter strand 207a′ also includes, as part of the bridge region 208, replicated ssDNA daughter strand bridge portion 208a. Further, Y-branch element 213a remains at the 5′ end of displaced template strand 207a.

Likewise, polymerase 210b extends 3′ end of nick site 201b while also displacing 5′ end of nick site 201b (and its associated parental template strand 207b), using parental strand 207a as a template (FIG. 2C, lower panel). Hence, as polymerase 210b proceeds at Step 2c (lower panel), it extends 3′ end of nick site 201b using parental strand 207a as a template to synthesize the new, daughter strand 207b′ that is complementary to the sequence of parental strand 207a (and that hence shares the same sequence of parental strand 207b). As shown, Y-branch element 213b also remains attached to displaced parental strand at the 5′ end. The new daughter strand 207b′ also includes, as part of the bridge region 208, replicated ssDNA daughter strand bridge portion 208b.

Still continuing with the above example embodiment, FIG. 2D is a schematic depicting continued polymerase extension of the circular construct and formation of the double-length DNA template using the YBEA 200, in accordance with certain example embodiments. As illustrated in FIG. 2D (top panel), polymerase 210a continues along parental template strand 207b to 5′ end of parental template strand 207b, completing the synthesis of new daughter strand 207a′. Likewise, as illustrated in FIG. 2D (top panel), polymerase 210b continues along parental template strand 207a to 5′ end of parental template strand 207a, completing the synthesis of new daughter strand 207b′. Notably, new daughter strand 207a′ includes a Y-branch element daughter strand 213b′, the Y-branch element daughter strand 213b′ being complementary to Y-branch element 213b that is attached to parental strand 207b. Likewise, new daughter strand 207b′ includes a Y-branch element daughter strand 213a′, Y-branch element daughter strand 213a′ being complementary to Y-branch element 213a attached to parental strand 207a.

At Step 2d of FIG. 2D, once first and second polymerases 210a and 210b complete synthesis of daughter strands 207a′ and 207b′, respectively, polymerases 210a and 210b dissociate from the circular construct 209, forming the double-length DNA template 211 (FIG. 2D, lower panel). As illustrated in FIG. 2D (lower panel), the double-length DNA template 211 includes two copies of the target DNA template 207, i.e., first and second copies 211a and 211b, respectively, each flanking the bridge region 208 (the bridge being derived from the YBEA 200 and including bridge daughter strand portions 208a and 208b).

As shown, each template copy 211a and 211b includes both a parental polynucleotide strand (shown in black) and newly synthesized daughter polynucleotide strand (shown in gray). For example, template copy 211a includes original (parental) template strand 207b and newly synthesized daughter strand 207a′. On the other side of the bridge region 208, template copy 211b includes original (parental) template strand 207a and newly synthesized daughter strand 207b′. Further, the double-length DNA 211 template includes a first terminal end 212a and a second terminal end 212b. The first terminal end 212a, for example, includes the portion of the YBEA 200 strand 200b associated with 5′ end of nick site 201b YBEA 200 (open black rectangle at terminal end 212a) and its copy (open gray circles at terminal end 212a). Likewise, second terminal end 212b of the double-length DNA template 211 includes the portion of the YBEA 200 strand 200a associated with 5′ end of nick site 201a YBEA 200 (open black circles at 212b) and its copy (open gray rectangle at terminal end 212b).

As is also shown, template copy 211a also includes at terminal end 212a Y-branch element 213b and its complementary sequence in Y-branch element daughter strand 213b′, while template copy 211b includes at terminal end 212b Y-branch element 213a and its complementary sequence in Y-branch element daughter strand 213a′ (FIG. 2D, lower panel). That is, the Y-branch elements 213a and 213b of the YBEA 200 are present at the terminal ends of the double-length DNA template 211, as derived from the YBEA 200 as described herein. In this way, combination of the YBEA 200 with a parental target DNA template 207 via Steps 2a-2d of FIGS. 2B-2D yields a double-length DNA template 211 (FIG. 2D, lower panel) that includes a predetermined oligonucleotide sequence at each end.

Further, and similar to the example double-length DNA template 111 shown in FIG. 1D (lower panel), both strands of the double-length DNA template 211 of FIG. 2D also include a parental polynucleotide strand (in black) covalently joined to a newly synthesized daughter copy (in gray) of the target DNA template. For example, parental strand 207a is covalently and contiguously joined to newly synthesized daughter strand 207a′ via a strand of the bridge region 208 (i.e., the strand of the bridge region 208 including strand portions 200a and 208a) in a 5′→3′ direction. Further, because of the polymerase-mediated extension of the circular construct as described herein, the nucleotide sequence of parental strand 207a matches that of new daughter stand 207a′. That is, daughter strand 207a′ is a sequence copy of the parental strand 207a of the target DNA template.

Likewise, parental strand 207b is covalently and contiguously joined to new daughter strand 207b′ via a strand of the bridge region 208 (i.e., the strand of the bridge region 208 including strand portions 200b and 208b), also in a 5′→3′ direction. And similarly, the nucleotide sequence of parental strand 207b matches that of new daughter stand copy 207b′. That is, daughter strand 207b′ is a sequence copy (i.e., a daughter copy) of the parental strand 207b of the target DNA template. In this way, each strand of the double-length DNA includes both includes both a parental polynucleotide sequence and a daughter polynucleotide sequence copy on each strand, in addition to the parental template strand and its complementary daughter strand on each of the two target DNA copies 211a and 211b (FIG. 2D, lower panel).

As described herein, in certain example embodiments the Y-branch elements 213a and 213b can be used to facilitate amplification, such as PCR amplification. Accordingly, FIG. 2E is an illustration showing the double-length DNA template 212 of FIG. 2D (lower panel) in a denatured (single-stranded) form, in which the original Y-branch elements 213a and 213b provide a predetermined oligonucleotide primer binding sequence, when replicated, in accordance with certain example embodiments. For example, when Y-branch element 213a is replicated as 213a′ (FIG. 2D), the 213a′ daughter Y-branch element includes a primer binding site at or within Y-branch element 213a′. Likewise, when Y-branch element 213b is replicated as 213b′ (FIG. 2D), the 213b′ daughter Y-branch element includes a primer binding site at or within Y-branch element 213b′. As shown in FIG. 2E, following a PCR denaturing step, primer 214 binds the primer binding site of Y-branch element 213a′, while primer 215 binds the primer binding site Y-branch element of 213b′. And as with conventional PCR protocols and procedures, the primers 214 and 215 provide a 3′ end for polymerase extension. In this way, the YBEA 200 embodiment can be used to duplicate a template strand and then provide primer binding sites for conventional PCR-based template amplification.

In certain example embodiments, primers 214 and 215 have the same sequence and hence bind the same sequence within their respective primer binding sites of Y-branch elements 213a′ and 213b′. That is, the primers 214 and 215 are the same. Alternatively, in certain example embodiments primers 214 and 215 have different sequences and hence bind to different sequences within their respective primer binding sites of Y-branch elements 213a′ and 213b′. Hence, the YBEA—and its associated Y-branch elements—provide a unique ability to customize replication of a target DNA template strand for downstream applications, such as PCR amplification.

### Epigenetic Analyses Using Double-Length DNA Templates

As described herein, in certain example embodiments the target DNA template includes the native, target sequence. As such, in certain example embodiments the target DNA template can retain epigenetic information regarding the target sequence, such as a methylation pattern of the target sequence. And because parental polynucleotide strands of the target DNA template are retained in the double-length DNA template as described herein, i.e., each strand of the double-length DNA template includes a parental polynucleotide strand from the target DNA template (referred to as the “parental copy” of the target sequence in the context of the double-length DNA template), the double-length DNA template also preserves epigenetic information from the target sequence.

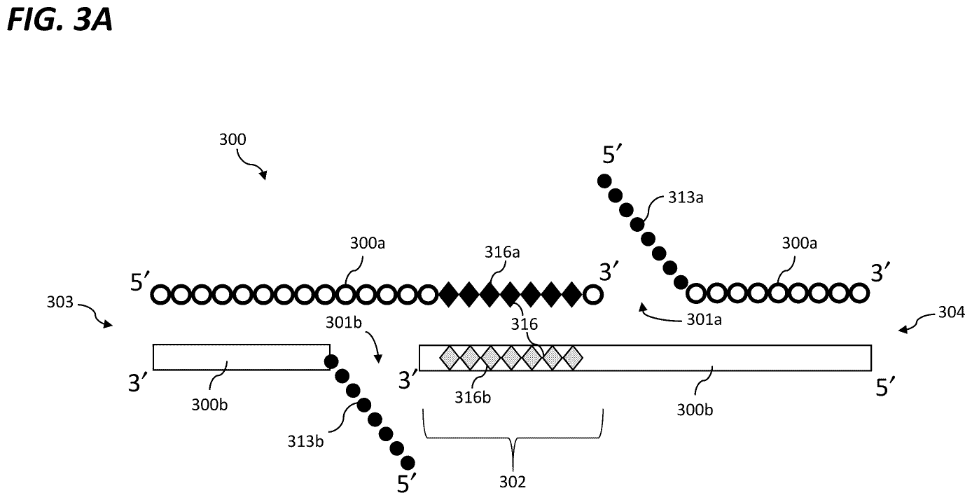

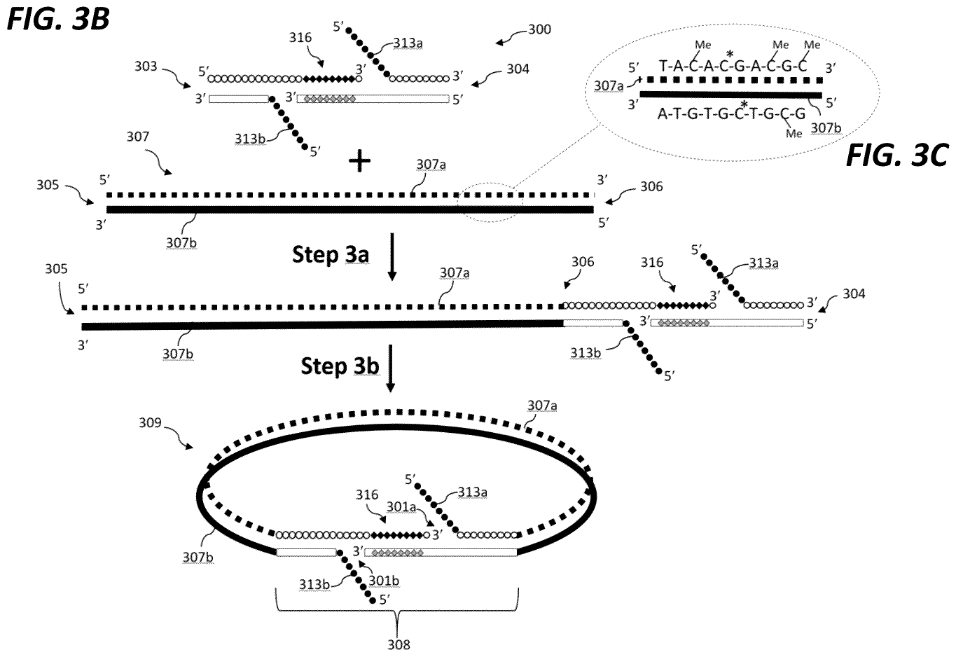

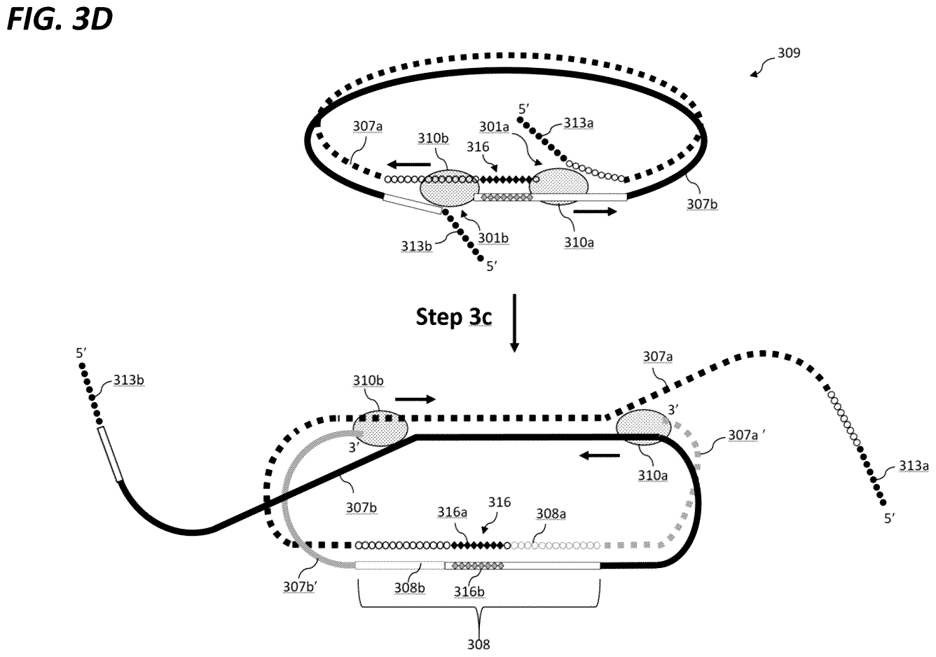

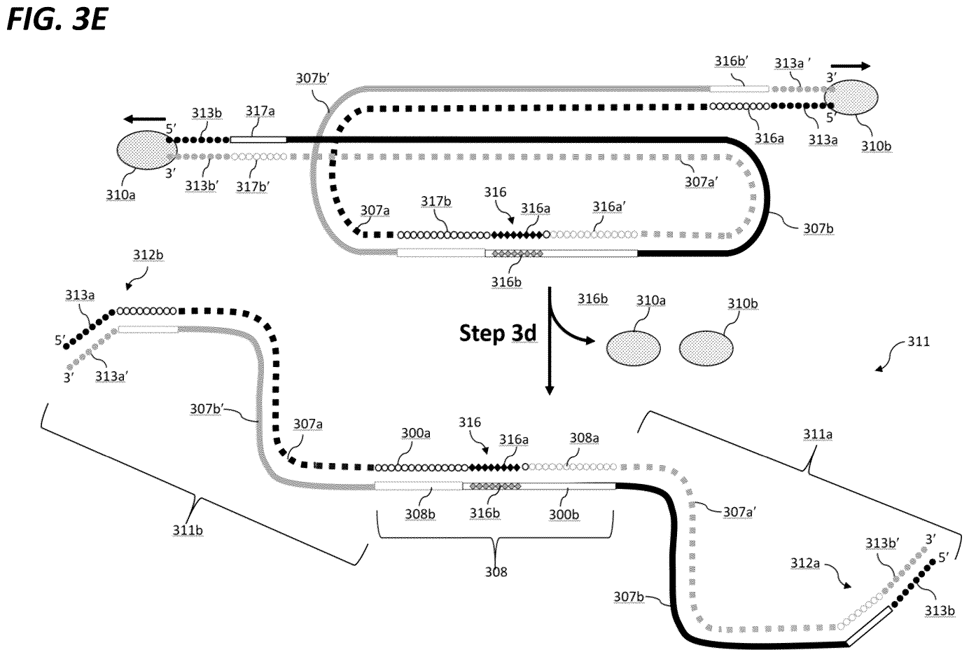

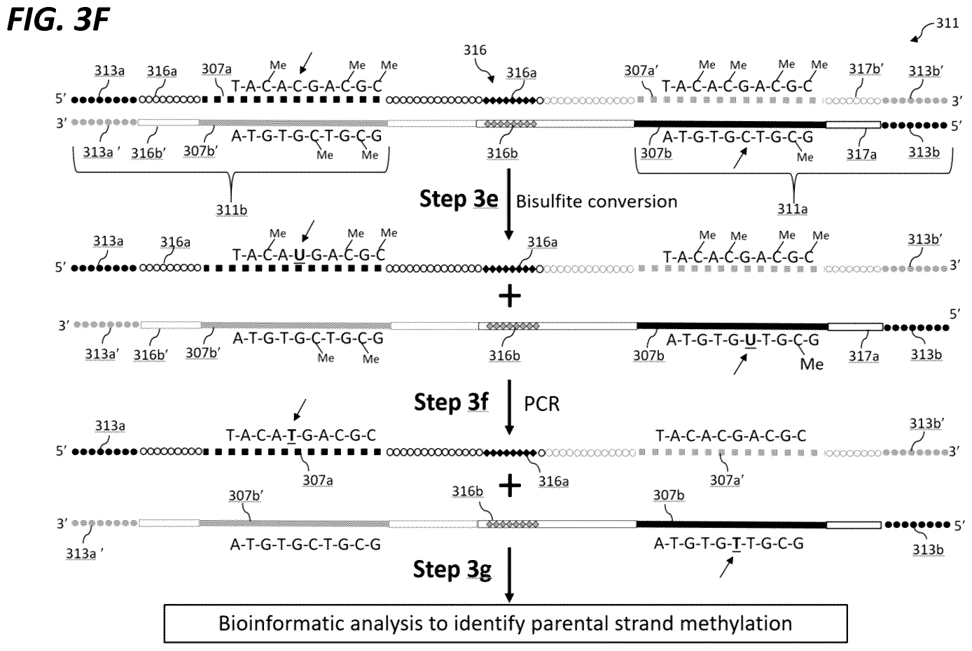

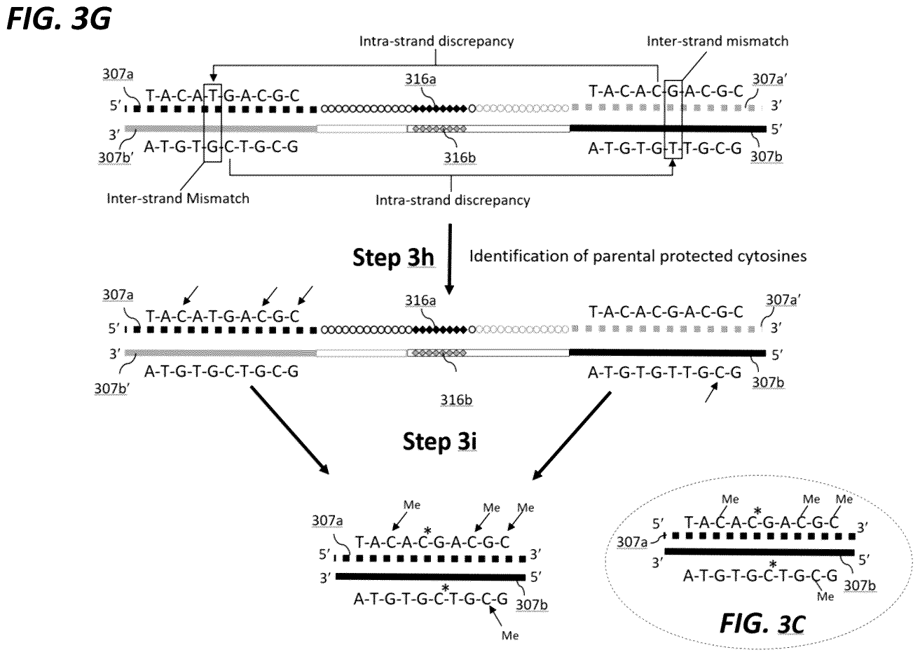

Further, the daughter copies of the target sequence can be synthesized under conditions that preserve the genetic information of the target sequence, as described further herein. The presence of both a parent copy and a daughter copy of the target sequence on the same strand of the double-length DNA template is thus particularly beneficial for “intra-strand” comparisons to discern epigenetic information. And because each parental copy of the target DNA template in the double-length DNA template is also hybridized to a complementary daughter sequence, in certain example embodiments this arrangement also permits “inter-strand” comparisons to discern epigenetic information. The dual means of comparing parental and daughter sequences advantageously increases the accuracy of—and confidence in—the epigenetic information detected in the target sequence. These and other example embodiments are illustrated and described with regard to FIGS. 3A-3G.

To facilitate intra-strand and/or inter-strand comparisons, in certain example embodiments the end adapters provided herein, such as the YBEA of FIG. 2A, can be further modified to provide features that enable bioinformation grouping of sequence reads. For example, the end adapter, such as the YBEA of FIG. 2A, can be modified to include a unique molecule identifier (UMI). In certain example embodiments, the UMI can be included within the spacer region 202 of the YBEA, in which case the UMI sequences of the two strands of the double-length DNA template will have reverse complement sequences. Such a modified end adapter—and its use in a method for performing epigenetic analyses of the target DNA template strands and hence the target sequence—is shown in FIGS. 3A-3F.

With reference to FIG. 3A, provided is an illustration showing a Y-Branch End Adapter that includes a UMI (“YB-UMI-EA”), in accordance with certain example embodiments. As shown, the YB-UMI-EA 300 has the general duplexed polynucleotide YBEA structure as shown in FIG. 2A. This includes, for example, hybridized strands 300a and 300b, where “300a” refers to the entire 5′→3′ strand of the YBEA 300 (with the nick site 301a within the strand 300a) and “300b” refers to the entire 5′→3′ strand hybridized to strand 300a (with the nick site 301b within the strand 300b). Further, single-stranded Y-branch elements 313a and 313b attached to the 5′ end of the first and second nick sites 301a and 301b, respectively, of the YB-UMI-EA. Y-branch element 313a and 313b, for example, can include a predetermined oligonucleotide sequence, the design of which can be tailored to achieve a specific objective, such as, e.g., PCR amplification, as described above with regard to FIGS. 2A-2E.

For example, and as shown in FIG. 3A, each Y-branch element 313a and 313b can include a predetermined oligonucleotide sequence that provides a complementary or hybridizable primer binding site useful for PCR amplification of a new daughter strand. That is, each Y-branch element 313a and 313b, for example, can include 10-30 nucleotides, such as 15-25 nucleotides or 18-22 nucleotides, the complement of which includes a primer binding site sequence. In certain example embodiments, the Y-branch element 313a and 313b include the same sequence, as indicated in FIG. 3A (at 313a and 313b). Alternatively, the Y-branch element 313a and 313b include different sequences. In certain example embodiments, the Y-branch element 313a and 313b are the same length, while in other example embodiments the Y-branch element 313a and 313b may be different lengths.

The YB-UMI-EA 300 also includes terminal ends 303 and 304 flanking each nick site, each end 303 and 304 being compatible with efficient ligation to the ends of a target DNA template. Preferably, the nick sites 301a and 301b are spaced apart by double-stranded spacer region 302 such that the EA can accommodate attachment of two polymerases for bidirectional extension, as described herein. That is, the nick sites 301a and 301b are far enough apart such that binding of one polymerase does not sterically hinder and/or displace the binding of a second polymerase. This is shown in FIG. 3A, where the spacer region 302 linearly offsets first nick site 301a from the second nick site 301b.

As is also shown, positioned within the spacer region 302 of the YB-UMI-EA 300, for example, is a UMI sequence 316. The UMIs, also known as molecular barcodes or random barcodes, include short, random and/or predetermined nucleotide sequences that are incorporated into an oligonucleotide sequence. Typically, UMIs are 5-20 nucleotides in length, such as 8-16 nucleotides. Of course, this length can vary depending on the application. For example, the UMI can have a length of at least of 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides. More conventionally, the UMI includes a sequence of 5, 6, 7, 8, 9, 10, 11, 12, 13, 14, 15, 16, 17, 18, 19, 20, 21, 22, 23, 24, or 25 nucleotides.

As shown in FIG. 3A, because the YB-UMI-EA 300 is double stranded, UMI sequence 316 has a complementary UMI strand sequences 316a and 316b, with strand 316a shown in a 5′→3′ polarity and the 316b strand having the complementary 3′→5′ polarity. Further, because UMI strand sequences 316a and 316b are complementary sequences, the sequences of the different strands of the resultant double-length DNA template can be bioinformatically paired to enable comparison sequencing reads of different strands as described herein.

With reference to FIG. 3B, provided is a schematic showing circularization of a target DNA template using the YB-UMI-EA 300, in accordance with certain example embodiments. As shown, the YB-UMI-EA 300, with its respective first and second single-stranded Y-branch elements 313a and 313b and double-stranded UMI sequence 316, is combined with a double-stranded target DNA template 307 to form a circular construct 309. That is, the YB-UMI-EA 300 is combined with a target DNA template 307, the template 307 having first and second terminal ends 305 and 306 and including complementary strands 307a and 307b. At Step 3a, for example, the YB-UMI-EA 300 is ligated to either end of the target DNA template 307, the target DNA template 307 including polynucleotide strands 307a and 307b. For example, terminal end 303 of the YB-UMI-EA 300 is ligated to template end 306. Alternatively at Step 3a, and though not shown for simplicity, the other terminal end of the YB-UMI-EA 300 (i.e., 304) is ligated to terminal end 305 of the target DNA template 307.

At Step 3b of FIG. 3B, the un-ligated (free) end of the YB-UMI-EA 300 is ligated to the other free end of the target DNA template 307 to form a circular construct 309 (or circular construct). For example, if terminal end 303 of the YB-UMI-EA 300 is ligated to template end 306 at Step 3a, then at Step 3b terminal end 304 of YB-UMI-EA 300 is ligated to template terminal end 305, thereby forming a circular construct 309 of the original (parental) target DNA template 307. Alternatively, if terminal end 304 of the YB-UMI-EA 300 is ligated to template end 305 at Step 3a, then at Step 3b terminal end 303 is ligated to template terminal end 306, thereby forming the circular construct 309.

Either way, at Step 3b of FIG. 3B, the two terminal ends 303 and 304 of the YB-UMI-EA 300 join each end 305 and 306 of the template 307, thereby forming a YB-UMI-EA bridge region 308 between the ends of the template 307. That is, the entirety of the YB-UMI-EA 300 forms the bridge region 308 between the two ends 305 and 306 of the target (parental) template 307. This forms the circular construct 309 (or circular construct) that includes the YB-UMI-EA 300 (as bridge region 308) along with complementary parental template strands 307a and 307b of the parental template 307. In this way, the YB-UMI-EA 300 (FIG. 3A) operates as bridge precursor for the bridge region 308. Further, the respective first and second nick sites 301a and 301b remain in the circular construct 309 (as part of the bridge region 308) and hence provide a two respective 3′ ends available for polymerase attachment and bidirectional extension, as described herein. The single-stranded Y-branch elements 313a and 313b are also present in the circular construct 309, along with UMI sequence 316.

With reference to FIG. 3C, provided is an enlarged view of a portion of the target DNA template 307, showing an example nucleic acid sequence, in accordance with certain example embodiments. As shown, the portion of target DNA template strand 307a includes endogenously methylated cytosine residues (i.e., 5-Methylcytosine or “5mc”) at nucleotide positions 3, 8, and 10 of the example sequence and an unmethylated cytosine residue (arrow) at position 5 (when strand 307a is read from left to right, i.e., in 5′→3′ direction for strand 307a). The cytosine residue at the fifth position (see asterisk in strand 307a), however, is unprotected (i.e., unmethylated). Further, target DNA template strand 307b, which is complementary to target DNA template strand 307a, includes an endogenously methylated cytosine residue at position 9 (when from left to right, i.e., 3′→5′ direction for strand 307b). The cytosine residue at the sixth position (see asterisk is strand 307b) is unprotected (i.e., unmethylated). In this example, the 5mC residues represent an epigenetic methylation pattern of a parental target DNA template target.

Continuing with the YB-UMI-EA 300 example embodiment, FIG. 3D is a schematic depicting polymerase attachment and initiation of bidirectional extension of a circular construct including the YB-UMI-EA 300, in accordance with certain example embodiments. As illustrated in FIG. 3D (and akin to FIG. 2C), when first and second polymerases 310a and 310b are combined with the DNA circular construct 309, they bind to nick sites 301a and 301b, respectively, and proceed in opposite directions (as indicated by the arrows in FIG. 3D, top panel). The polymerases 310a and 310b also displace 5′ end of parental strands 307a and 307b, respectively (including their associated and respective 313a and 313b Y-branch elements). The UMI strand sequences 316a and 316b of UMI 316 remain unaltered.

At Step 3c (FIG. 3D, lower panel), polymerases 310a and 310b bidirectionally extend the circular construct 309 in opposite directions (see arrows). That is, as polymerase 310a proceeds, it extends 3′ end of nick site 301a using parental strand 307b as a template to synthesize the new, daughter strand 307a′ that is complementary to the sequence of parental strand 307b (and that hence shares the same sequence of parental strand 307a). The new daughter strand 307a′ also includes, as part of the bridge region 308, replicated ssDNA daughter strand bridge portion 308a. Further, Y-branch element 313a remains at the 5′ end of displaced template strand 307a, while UMI sequence 316, with its strand sequences 316a and 316b, remains unchanged.

Likewise, polymerase 310b extends 3′ end of nick site 301b while also displacing 5′ end of nick site 301b (and its associated parental template strand 307b), using parental strand 307a as a template (FIG. 3D, lower panel). Hence, as polymerase 310b proceeds at Step 3c (lower panel), it extends 3′ end of nick site 301b using parental strand 307a as a template to synthesize the new, daughter strand 307b′ that is complementary to the sequence of parental strand 307a (and that hence shares the same sequence of parental strand 307b). As shown, Y-branch element 313b also remains attached to displaced parental strand 307b at the 5′ end. The new daughter strand 307b′ also includes, as part of the bridge region 308, replicated ssDNA daughter strand bridge portion 308b. And again, UMI sequence 316, with its strand sequences 316a and 316b, remains unchanged as it is not replicated during strand extension.

With reference to FIG. 3E, provided is a schematic depicting continued polymerase extension of the circularized target DNA template and formation of the double-length DNA template using the YB-UMI-EA 300 example embodiment, in accordance with certain example embodiments. As illustrated in FIG. 3E (top panel), polymerase 310a continues along parental template strand 307b to 5′ end of parental template strand 307b, completing the synthesis of new daughter strand 307a′. Likewise, as illustrated in FIG. 3E (top panel), polymerase 310b continues along parental template strand 307a to 5′ end of parental template strand 307a, completing the synthesis of new daughter strand 307b′. Notably, new daughter strand 307a′ includes a Y-branch element daughter strand 313b′, the Y-branch element daughter strand 313b′ being complementary to Y-branch element 313b that is attached to parental strand 307b. Likewise, new daughter strand 307b′ includes a Y-branch element daughter strand 313a′, Y-branch element daughter strand 313a′ being complementary to Y-branch element 313a attached to parental strand 307a. The UMI sequence 316, with its strand sequences 316a and 316b, remains unchanged.

At Step 3d of FIG. 3E, once first and second polymerases 310a and 310b complete synthesis of daughter strands 307a′ and 307b′, respectively, the polymerases 310a and 310b dissociate from the circular construct 309, forming the double-length DNA template 311 (FIG. 3E, lower panel). As illustrated in FIG. 3E (lower panel), the double-length DNA template 311 includes two copies of the original (parental) target DNA template 307, i.e., first and second copies 311a and 311b, respectively, each flanking the bridge region 308. The bridge region 308, which is derived from the YB-UMI-EA 300, includes bridge daughter strand portions 308a and 308b, along with the unchanged UMI 316 and its complementary strand sequences 316a and 316b.

As shown, each template copy 311a and 311b includes both a parental polynucleotide strand and newly synthesized daughter polynucleotide strand. For example, template copy 311a includes original (parental) template strand 307b of the target DNA template 307 and newly synthesized daughter strand 307a′ (FIGS. 3B and 3G). On the other side of the bridge region 308, template copy 311b includes original (parental) template strand 307a of target DNA template 307 and newly synthesized daughter strand 307b′ (FIGS. 3B and 3G). Further, the double-length DNA 311 template includes a first terminal end 312a and a second terminal end 312b. The first terminal end 312a, for example, includes the portion of the YB-UMI-EA 300 strand 300b associated with 5′ end of nick site 301b YB-UMI-EA 300 (open black rectangle at terminal end 312a) and its copy (open gray circles at terminal end 312a). Likewise, second terminal end 312b of the double-length DNA template 311 includes the portion of the YB-UMI-EA 300 strand 300a associated with 5′ end of nick site 301a YB-UMI-EA 300 (open black circles at 312b) and its copy (open gray rectangle at terminal end 312b).

As is also shown, template copy 311a also includes at the first terminal end 312a Y-branch element 313b and its complementary sequence in Y-branch element daughter strand 313b′, while template copy 311b includes at the second terminal end 312b Y-branch element 313a and its complementary sequence in Y-branch element daughter strand 313a′ (FIG. 3E, lower panel). In this way, combination of the YB-UMI-EA 300 with a target DNA template strand 307 via Steps 3a-3d of FIGS. 3B-3D yields a double-length DNA template 311 (FIG. 3E, lower panel) that includes a predetermined oligonucleotide sequence at each end, along with a UMI 316 (and its strand sequences 316a and 316b).

As with the double-length DNA templates 111 and 211 of FIGS. 1D and 2D, both strands of the double-length DNA template 311 shown in FIG. 3D also include a parental copy (in black) joined to a newly synthesized daughter copy (in gray) of the target DNA template. For example, parental copy 307a is covalently and contiguously joined to newly synthesized daughter copy 307a′ via a strand of the bridge region 308 (i.e., the strand of the bridge region 308 including strand portions 300a and 308a) in a 5′→3′ direction. Further, because of the polymerase-mediated extension of the circular construct as described herein, the nucleotide sequence of parental copy 307a matches that of new daughter copy 307a′.

Likewise, parental copy 307b is covalently and contiguously joined to new daughter copy 307b′ via a strand of the bridge region 308 (i.e., the strand of the bridge region 308 including strand portions 300b and 308b), also in a 5′→3′ direction. And similarly, the nucleotide sequence of parental copy 307b matches that of new daughter copy 307b′. In this way, each strand of the double-length DNA includes both parental template DNA and daughter copy DNA on each strand, in addition to parental template DNA hybridized to complementary daughter DNA in each of the two target DNA copies (FIG. 3E, lower panel).

In certain example embodiments, the double-length DNA template of FIG. 3E (bottom panel)—can advantageously be used to discern epigenetic information associated with the parental target DNA template 307. For example, during the polymerase 310a and 310b extension steps described in FIG. 3E-3F, protected nucleotides, such as methylated cytosine nucleotide residues, can be used for daughter strand extension and synthesis. This results in incorporation of the protected nucleotide, such as the methylated cytosine nucleotide residue, into the newly synthesized daughter strands 307a′ and 307b′. In such embodiments, the daughter strand—with the protected cytosine residues—preserves the genetic information of the target DNA template during, e.g., a bisulfite treatment process, which converts natural cytosine to uracil. Thereafter, following a bisulfite conversion reaction and DNA sequencing, as described further herein, bioinformatic analysis of the sequence information can be performed to identify methylated cytosine residues in the original (parental) target DNA template.

In certain example embodiments, the identification of methylated cytosine residues in the original (parental) target DNA template 307 provides epigenetic information associated with the original (parental) target DNA template 307. This is shown, for example, in FIG. 3F, which provides a schematic showing an example bisulfite conversion of the double-length DNA template 311 and thereafter its PCR-amplified products, via the use of the Y-branch end adapter with a UMI (i.e., YB-UMI-EA 300) of FIG. 3A, in accordance with certain example embodiments.

With reference to FIG. 3F (and before Step 3e), a truncated example portion of the double-length DNA 311 of FIG. 3E is shown, the truncated portion including a double copy of the example template sequence portion shown in FIG. 3C. That is, FIG. 3F shows only a portion of the sequence of the original target DNA template 311 (for simplicity of illustration only), the portion shown including two copies of the example sequence (311a and 311b) according to FIG. 3C. As shown, each strand includes, in an intra-strand arrangement, a parent copy (black) and daughter copy (gray), for example, the copies resulting from the formation of the double-length DNA template as described herein.

As is also shown in FIG. 3F, each template copy portion 311a and 311b includes 10 example nucleotide pairs, corresponding to those of FIG. 3C, the nucleotide sequence being mirrored on each side of the double-length DNA template as a consequence of forming the double-length DNA template. For example, the sequence of parental strand 307a (of template copy 311b) corresponds to the same sequence on daughter strand 307a′ (of template copy 311a), with both sequences 307a and 307a′ being associated with UMI strand sequence 316a. For example, reading the polynucleotide sequence associated with UMI strand 316a from to left right (i.e., 5′→3′), the example parental copy (in black) is TACACGACGC (SEQ ID NO: 1), while the daughter copy (in gray) is the same polynucleotide sequence, i.e., TACACGACGC. Hence, the example 5′→3′ sequence associated with UMI 316a is TACACGACGC-UMI-TACACGACGC.

Likewise, given the complementary base-pairing of the strands, the sequence of parental strand 307b (of template copy 311a) corresponds to the same sequence on daughter strand 307b′ (of template copy 311b), but with both sequences 307b and 307b′ being associated with UMI strand sequence 316b. That is, reading the sequence associated with UMI strand 316b from left to right (i.e., 3′→5′), the example daughter strand sequence (in gray) is ATGTGCTGCG (SEQ ID NO:2) while the parental sequence (in black) is also ATGTGCTGCG. In other words, the example 5′→3′ sequence associated with UMI 316b is ATGTGCTGCG-UMI-ATGTGCTGCG. As such, each UMI strand sequence 316a and 316b of UMI 316 is associated with a portion of a parental strand (in black) and a new daughter strand (in gray) (FIG. 3F).

As is also shown in this example epigenetic evaluation of the target DNA template strand, before Step 3e parental strand 307a of template copy 311b includes endogenously methylated (protected) cytosine residues at positions 3, 8, and 10, with an unmethylated cytosine residue (arrow) at position 5 (from left to right, i.e., 5′→3′, and as is also shown in FIG. 3C). Further, parental strand 307b of template copy 311a includes endogenously methylated (protected) cytosine residue at position 9, with an unmethylated residue at position 6 (as is also shown in FIG. 3C, when read from 3′→5′). Each daughter strand 307a′ and 307b′, however, includes only methylated cytosine residues as a consequence of polymerase extension with only methylated cytosine residues provided in the extension reaction. That is, neither of the daughter strands 307a′ or 307b′ include an unmethylated cytosine residue. As such, the protected daughter strands 307a′ or 307b′ (in gray) preserve the genetic information of the target DNA template, while the native parent target DNA template strands 307a and 307b (in black) preserve the epigenetic information of the target DNA template (and hence of the target sequence).

At Step 3e of FIG. 3F, the double-length DNA template is subjected to bisulfite conversion using conventional methodologies. For example, bisulfite conversion is a method that uses bisulfite to determine the methylation pattern of DNA, such as the methylation of a target DNA template. DNA methylation, for example, is an endogenous biochemical process involving the addition of a methyl group to the cytosine or adenine DNA nucleotides. DNA methylation, for example, stably alters the expression of genes in cells as cells divide and differentiate from embryonic stem cells into specific tissues. In bisulfite conversion (also known as bisulfite sequencing), target nucleic acids are first treated with bisulfite reagents that specifically convert un-methylated cytosine residues to uracil residues (i.e., a C→U conversion), while having no impact on methylated cytosine residues (i.e., the methylated cytosine residues are “protected” from the C→U conversion). Thereafter, a PCR reaction with native adenine (A), cytosine (C), guanine (G), and thymine (T) nucleotides substitutes the converted uracil residue with a thymine residue (i.e., a U→T substitution). In this way, unmethylated (i.e., “unprotected”) cytosine residues are converted to a thymine via an intermediate uracil (i.e., C→U→T).

Accordingly, as shown at Step 3e of FIG. 3F, subjecting the strands of the denatured double-length DNA template to a bisulfite conversion reaction causes conversion of the unmethylated cytosine residue at position 5 of parental strand 307a to a uracil residue (as shown with bold and underlined, SEQ ID NO:3), i.e., a 5C→5U conversion. Likewise, the unmethylated cytosine residue at position 6 of parental strand 307b is converted to a uracil (as shown with bold and underlined, SEQ ID NO: 4), i.e., a 6C→6U conversion. The bisulfite reaction, however, does not affect any of the methylated (protected) cytosine residues in parental strands 307a and 307a, i.e., these cytosine residues remain cytosine residues. This includes the daughter strand 307a′ and 307b′ methylated cytosine residues that were included within the daughter strands 307a′ and 307b′ via polymerase extension using methylated cytosine nucleotides, as described herein.

Following the bisulfite conversion reaction of Step 3e, at Step 3f of FIG. 3F the bisulfite converted strands of the denatured double-length DNA template 311 product are subjected to PCR amplification and sequencing reactions using conventional methodologies. For example, PCR primers directed to the Y-branch elements 313a′ and 313b′ can be used to amplify the strands of the denatured double-length DNA template 311, such as described herein. As shown at Step 3f, and as can be determined via conventional DNA sequencing, the PCR product (shown in a denatured state for illustration purposes) yields distinct strands, each associated with either UMI strand sequence 316a or 316b. In the PCR product, the uracil residues produced by bisulfite conversion of the unmethylated (unprotected) cytosine residues are substituted with thymine. For example, the uracil residue at position 5 of parental strand 307a is substituted with a thymine (T) residue during the PCR reaction (see arrows), i.e., a 5U→5T substitution. Further, the 5U→5T substitution of parental strand 307a is associated with UMI strand sequence 316a. Likewise, the uracil residue at position 6 of parental strand 307b is substituted with a thymine (T) residue during the PCR reaction (see arrows), i.e., a 6U→6T conversion. Further, the 6U→6T substitutions of parental strand 307b is associated with UMI strand sequence 316a. As such, each strand of the UMI (316a and 316b) in this example localizes with strand-specific nucleotide conversions (C→U→T) of the original (parental) DNA template.

At Step 3g, following the PCR reaction of Step 3f the PCR products are sequenced, the resulting sequencing reads identifying the methylation pattern of the original parental copies of the DNA target sequence through intra-strand comparison of parent and daughter sequences. That is, the daughter strand copy, with protected cytosine residues, is resistant to bisulfite conversion and thus preserves the genetic sequence of the parent template. Hence, at each position in which the original parental strand sequence includes a native (unmethylated) cytosine, the sequence read of the entire strand will indicate a discrepancy between the parent and daughter sequences; in contrast, at each position in which the parent strand sequence includes a methylated cytosine, the sequence read of the entire strand will show accordance between the parent and daughter sequences

Additionally or alternatively, comparison of the sequences of complementary parental-derived and daughter strands (i.e., inter-strand comparison) can be used to also identify and/or confirm the parental sequence methylation pattern. That is, comparison of parental-derived and daughter strand sequences of different strands of the double-length DNA template (enabled by bioinformatic grouping of UMI read sequences) will reveal mismatches between paired bases at the positions of native cytosine in the parent sequence, whereas positions of methylated cytosine will show normal complementarity to the daughter sequence. Such intra-strand and inter-strand comparisons and analyses are illustrated in FIG. 3G, with either method being used independently or in combination to assess epigenetic information associated with the original target template sequence.

With reference to FIG. 3G (before Step 3h), provided is a schematic showing both intra-strand and inter-strand comparison of a portion of the double-length DNA template to ascertain epigenetic information associated with the original target DNA template, in accordance with certain example embodiments. In this example schematic, the same example sequences are carried over from FIG. 3F, with the strands being shown in an aligned, double-length DNA double template configuration for illustration purposes only. As shown, for the intra-strand analyses, for example—when reading the sequence of the strand associated with UMI sequence 316a in 5′→3′ direction (i.e., left to right from the 5′ end of strand fragment 307a to the 3′ end of strand fragment 307a′)—an intra-strand T-C discrepancy is identified at the fifth nucleotide position (see arrow associated with UMI sequence 316a). That is, the sequence of strand fragment 307a (in black) includes a thymine (T) residue while the sequence of strand fragment 307a′ (in gray) includes a cytosine (C) residue. Importantly, because the daughter strands are synthesized with protected cytosine analogs, an intra-strand T-C discrepancy identifies strand fragment 307a′ as a daughter copy and fragment 307a as a parent copy of the target DNA template.

Likewise, in the strand associated with UMI sequence 316b, when reading the sequence of the strand associated with UMI strand 316b in 3′→5′ direction (i.e., left to right from 3′ end of strand fragment 307b′ to 5′ end of strand fragment 307b), an intra-strand T-C discrepancy is identified at the sixth nucleotide position (see arrow associated with UMI sequence 316b). That is, the sequence of strand fragment 307b (in black) includes a thymine residue, while the sequence of strand fragment 307b′ (in gray) includes a cytosine residue. And as with the T-C discrepancy associated with UMI sequence 316a discussed above, the presence of the cytosine residue at position six in strand fragment 307b′ identifies this strand fragment as a daughter strand (in grey), with strand fragment 307b (in black) being a parental-derived strand. Further, the presence of the substituted thymine residue at position six in strand 307b indicates, as described more fully below, that this thymine nucleotide was an unprotected cytosine residue in the original target sequence.

Additionally or alternatively, before step 3h, in certain example embodiments analyses of inter-strand mismatches can be used to identify, assess, and/or confirm the epigenetic information associated with the original target sequence. As shown, for example, inter-strand alignment of the sequence of example parental strand fragment 307a (in black) with the sequence of daughter strand fragment 307b′ (in gray) reveals a T-G mismatch at position 5 of the 307a/307b′ aligned sequences. And based on the presence of this mismatch, it can also be determined that the sequence of strand fragment 307a corresponds to a parental, target sequence. This is because only unmethylated (unprotected) cytosine residues undergo the C→U→T bisulfite/PCR conversion and because daughter strand extension with methylated (protected) cytosine residues only incorporates the protected cytosine residues into the daughter strand. Hence, only unprotected cytosine residues in the parental strands are converted to a thymine residue during the bisulfite/PCR conversion (i.e., not those of the daughter strand). Once strand fragment 307a is identified as a parental-derived copy, when reading from left to right (i.e., 5′→3′), this parental derived copy can be identified as associated with the 5′ end of UMI sequence 316a, with the daughter strand fragment 307a′ being positioned downstream from 3′ end of UMI 316a (as shown).

Likewise, alignment of the sequence of example parental strand fragment of 307b (in black) with the sequence of example daughter strand fragment 307a′ (in gray) reveals a T-G mismatch at position 6 of the 307b/307a′ aligned sequences. As such, the presence of the thymine residue in the T-G mismatch identifies strand fragment 307b as a parental-derived strand, with strand fragment 307a′ being a complementary daughter strand. Hence, once strand 307b is identified as the parental-derived copy, when reading from left to right (i.e., 3′→5′), this parental derived copy can be identified as associated with 3′ end of UMI strand fragment 316a, with the daughter strand fragment 307b′ being positioned upstream of 5′ end of UMI 316b, as shown. In certain example embodiments, using such inter-strand and intra-strand analysis can be used to identify and confirm methylation patterns across multiple sequence reads due to UMI-based read groupings. This is particularly beneficial, for example, where large regions of the target sequence—as preserved in the target DNA template—include methylated cytosine residues.

At Step 3h of FIG. 3G, based on the intra-strand and/or inter-strand analyses described herein, the protected (methylated) cytosine residues associated with the original (parental) target DNA template 307 can be identified. This in turn provides epigenetic information regarding the original (parental) DNA template 307. For example, as noted above the C→U→T bisulfite/PCR conversion only occurs with unprotected (native) cytosine residues. Hence, the presence of any cytosine residues in the strands identified as corresponding to the original (parental) target DNA template strands (i.e., strand fragments 307a and 307b in the above example, shown in black) can be identified as previously protected (methylated) cytosine residues. This is shown, for example, following Step 3h, where the arrows indicate the identification of cytosine residues that were protected in the original parental (target) template (e.g., template 307). As shown for strand fragment 307a, for example, previously protected cytosine residues are present at positions 3, 8, and 10 (see arrows, reading the strand from left to right). Similarly, the cytosine residue at position of 9 of strand fragment 307b can also be identified as previously protected (see arrow, reading from left to right).

Lastly, at Step 3i of FIG. 3G, in certain example embodiments the strand fragments identified as corresponding to the parental strands (e.g., 307a and 307b) of the original (parental) target DNA template 311—and their associated methylation pattern—can be aligned to reveal the epigenetic pattern associated with the original (parental) target DNA template 307. That is, by using the methods described in FIGS. 3A-3G, epigenetic information associated with the original (parental) target DNA template 307 can be obtained. As shown, for example, the aligned sequences of strand fragments 307a and 307b show methylation at positions 3, 8, and 10 of strand fragment 307a, as the presence of these cytosine residues in the strand corresponding to the parental template strand fragment 307a were necessarily protected (methylated) in the original (parental) template strand 307a (and hence not converted via bisulfite conversion). Further, the T-C discrepancy and/or the T-G mismatch, which identify the presence of a substituted thymine residue (because of the bisulfite conversion), can be used to assign a C residue at position five in place of the thymine residue in strand fragment 307a (see asterisk at fifth position cytosine residue of strand fragment 307a). Likewise, via this same or similar rationale, strand fragment 307b shows methylation at position 9 (reading left to right), with an unprotected cytosine (asterisk) at position six (when the sequence of strand fragment 307b is read left to right, i.e., 3′→5′). And notably, this identified epigenetic methylation pattern corresponds to the example methylation pattern provided as the example in FIG. 3C (see FIG. 3C inset).

Accordingly, by incorporating methylated cytosine nucleotides during the polymerase extension of the circularized target DNA template and thereafter subjecting the double-length DNA template to bisulfite/PCR conversion, epigenetic information associated with the original a target DNA template can be readily obtained by intra-strand and inter-strand parent/daughter sequence comparison.

In view of the disclosure herein, epigenetic detection methodologies can be incorporated into the methods of the present invention. For example, enzymatic conversion of modified bases of interest or any other biochemical or chemical reaction that specifically converts a modified nucleobase or interest relative to the native base (or, alternatively, converts an unmodified nucleobase of interest, as discussed herein in connection with bisulfite conversion of native cytosine to uracil). Certain example methods of enzymatic conversion of modified bases of interest are disclosed, e.g., in Applicants' co-pending U.S. Provisional Patent Applications Nos. 63/380,439 and 63/147,959, which are herein incorporated by reference in their entireties.

### Double-Length DNA Templates for Use in PCR Multiplexing

In certain example embodiments, the end adapter described herein can be additionally or alternatively modified to include one or more sequence indexes (SIDs). That is, the end adapter, such as the end adapters of FIGS. 1A, 2A, and/or 3A, can be modified to include one or more specific nucleotide sequences that identify, for example, the original source a target DNA template (and hence the target sequence) when multiple target DNA templates/target sequences are analyzed. Such SIDs, for example, are highly useful in applications such as DNA multiplexing, i.e., the processing of multiple, different samples at the same time, such as via PCR. Hence, SIDs are also referred to as sample identifiers.

In certain example embodiments, the same or different SIDs can be included adjacent to the Y-branch sequence elements described herein, such as in contiguous sequence with 3′ end of the Y-branch sequence elements described herein. Additionally or alternatively, one or more of the SIDs may be included on the same strand with a Y-branch sequence element, with an intervening non-SIDs nucleotide or series of nucleotides separating the SID from the Y-branch element. Regardless, each SID can be unique to a target sequence, with the complementary sequence to the SID found in the opposing (complementary) strand of the end adapter. Thereafter, double-length DNA molecules with different SIDs can be processed in a single PCR reaction, for example, the SIDs allowing differentiation of different DNA samples following sequencing. Further, because multiple copies of an SID will appear in a single, duplicated PCR product strand, bioinformatically the SID may be determined with high accuracy. This in turn reduces or eliminates the need for additional for error correction. In such example embodiments, the SIDs can also be used as landmarks in a given strand, allowing additional analytics. Further, such embodiments including SIDs can also include a UMI, such as described in FIGS. 3A-3E.

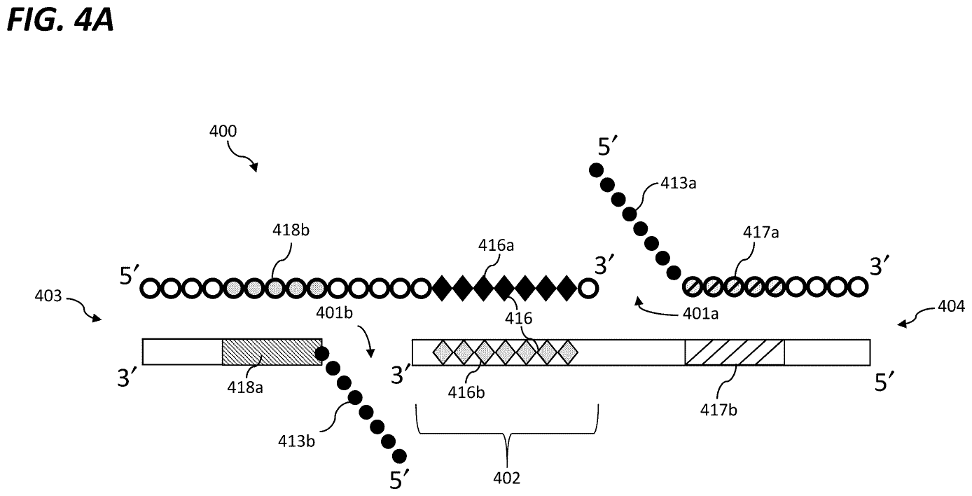

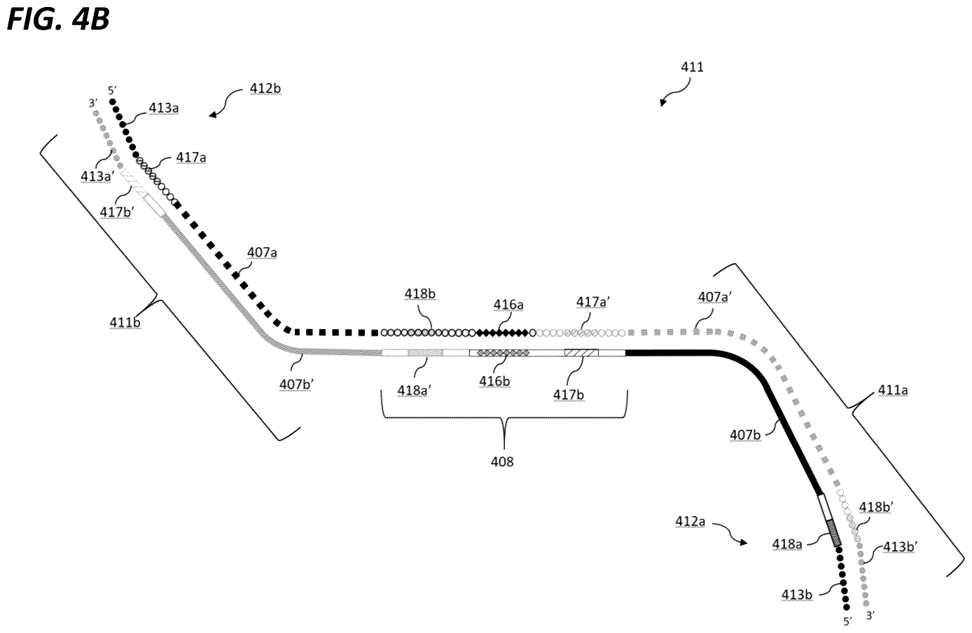

With reference to FIG. 4A, provided is an illustration of a Y-branch end adapter that includes two SID sequences and (optionally) a UMI, in accordance with certain example embodiments. As shown, the YB-UMI/SID-EA 400 has the general polynucleotide duplex YBEA structure as shown in FIG. 3A, including UMI 416 with UMI strands 416a and 416b. This includes, for example, Y-branch elements 413a and 413b attached to the 5′ end of the first and second nick sites 401a and 401b, respectively, of the YB-UMI/SID-EA. Y-branch element 413a and 413b, for example, can include a predetermined oligonucleotide sequence, the design of which can be tailored to achieve a specific objective, such as PCR amplification of the double-length DNA template, such as described above for FIGS. 2A-2E and FIGS. 3A-3E.

As shown in FIG. 4A, each Y-branch element 413a and 413b can include a predetermined oligonucleotide sequence that provides a complementary primer binding site useful for PCR amplification. That is, each Y-branch element 413a and 413b, for example, can include 10-30 nucleotides, such as 15-25 nucleotides or 18-22 nucleotides, the complement of which includes a primer binding site sequence. In certain example embodiments, the Y-branch elements 413a and 413b include the same sequence, as indicated in FIG. 4A (at 413a and 413b). Alternatively, the Y-branch element 413a and 413b include different sequences. In certain example embodiments, the Y-branch element 413a and 413b are the same length, while in other example embodiments the Y-branch element 413a and 413b may be different lengths.

The YB-UMI/SID-EA 400 also includes terminal ends 403 and 404 flanking each nick site, each end 403 and 404 being compatible with efficient ligation to the ends of a target DNA template. Preferably, the nick sites 401a and 401b are spaced apart by double-stranded spacer region 402 such that the EA can accommodate attachment of two polymerases for bidirectional extension, as described herein. That is, the nick sites 401a and 401b are far enough apart such that binding of one polymerase does not sterically hinder and/or displace the binding of a second polymerase. This is shown in FIG. 4A, for example, where the spacer region 402 linearly offsets the first nick site 401a from the second nick site 401b.

As is also shown, positioned within the spacer region 402 of the YB-UMI/SID-EA 400, for example, is a UMI sequence 416. Typically, UMIs are 5-20 nucleotides in length, such as 8-16 nucleotides. Of course, this length can vary depending on the application. For example, the UMI can have a length of at least of 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides. More conventionally, the UMI includes a sequence of 5, 6, 7, 8, 9, 10, 11, 12, 13, 14, 15, 16, 17, 18, 19, 20, 21, 22, 23, 24, or 25 nucleotides. As shown in FIG. 4A, because the YB-UMI/SID-EA 400 is double stranded, UMI sequence 416 has a complementary UMI strand sequences 416a and 416b, with strand 416a shown in a 5′→3′ polarity and the 416b strand having the complementary 3′→5′ polarity.

In addition to the UMI 416, which is shown in YB-UMI/SID-EA 400 but can be optionally included, YB-UMI/SID-EA 400 includes diagonally positioned SID 417a (gray circles with black crossline) and SID 418a (direction, shaded boxes), each shown contiguously joined to Y-branch elements 413a and 416b, respectively (solid black circles). That is, in the example shown in FIG. 4A, the sequence of each SID 417a and 418a occurs in series with 5′→3′ polynucleotide sequence of the Y-branch elements 413a and 413b, respectively. As is also shown, each of SIDs 417a and 418a have a respective complementary strand, i.e., SID complementary strands 417b (box with diagonal lines) and 418b (circles with gray fill).

Conventionally, SIDs include short, random and/or predetermined nucleotide sequences that can be incorporated into a polynucleotide sequence. Typically, SIDs are 5-20 nucleotides in length, such as 8-16 nucleotides. Of course, this length can vary depending on the application. For example, the SID can have a length of at least of 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides. More conventionally, the SID includes a sequence of 5, 6, 7, 8, 9, 10, 11, 12, 13, 14, 15, 16, 17, 18, 19, 20, 21, 22, 23, 24, or 25 nucleotides.

While YB-UMI/SID-EA 400 shows SIDs 417a and 418a located adjacent to and contiguously joined with Y-branch elements 413a and 413b, respectively, it is to be understood that one or more SIDs can be located anywhere in the YB-UMI/SID-EA 400 that facilitates sample differentiation. For example, one or more of the SIDs can be located contiguous with UMI strand 416a or 416b, such as on the 5′ side of UMI strand 416a or 5′ side of UMI strand 416b. In other example embodiments, the SIDs may be included within and/or as part of the UMI 416. Additionally or alternatively, one or more SIDs may be located on either end of the YB-UMI/SID-EA 400. For example, SID 417a may be located on 3′ end portion of terminal end 404 while SID 418a can be located 3′ end portion of terminal end 403. Hence, the SIDs described herein can be located at or within multiple and different locations of the YB-UMI/SID-EA 400, so long as the SID can allow sample differentiation as described herein.

With reference to FIG. 4B, shown is a double-length DNA template that arises from use of the YB-UMI/SID-EA 400 of FIG. 4A, in accordance with certain example embodiments. That is, the YB-UMI/SID-EA 400 is ligated to both ends of a target DNA template, thereby forming a circularized end adapter/target DNA template, such as is described in FIGS. 1B, 2B, and 3B. In other words, the same or similar steps described in FIGS. 1B, 2B, and 3B can be used to form a circular construct that includes the YB-UMI/SID-EA 400, the YB-UMI/SID-EA 400 forming a bridge that covalently links both ends of the target DNA template. Thereafter, using the same or similar steps as describe for FIGS. 1C-1D, 2C-2D, and 3D-3E, a first and second polymerase can be used to bind and extend 3′ ends of nick sites 401a and 401b of YB-UMI/SID-EA 400, such as in opposite directions, forming daughter strand copies as described herein. Once the polymerases complete their extensions, as described for example at Step 1d (FIG. 1D), Step 2d (FIG. 2D), and Step 3d (FIG. 3E), the polymerase dissociate from the circular construct, resulting in the double-length DNA template of FIG. 4B.

As shown in FIG. 4B, the double-length DNA template 411 includes two copies of the original (parental) DNA template 407, i.e., first and second copies 411a and 411b, respectively, each flanking the bridge region 408. The bridge region 408, which is derived from the YB-UMI/SID-EA 400, includes daughter strand SID sequence 417a′ and the complementary sequence 417b, with the daughter strand (in gray) formed via polymerase-mediated extension of 3′ end of nick site 401a. The bridge region 408 also includes daughter strand SID sequence 418a′ and the complementary sequence 418b, with the daughter strand (in gray) formed via polymerase-mediated extension of 3′ end of nick site 401b. The UMI 416 includes complementary UMI strand sequences 416a and 416b.

As shown, each DNA template copy 411a and 411b includes both a parental polynucleotide strand (in black) and newly synthesized daughter polynucleotide strand (in gray). For example, template copy 411a includes original (parental) template strand 407b and newly synthesized daughter strand 407a′. On the other side of the bridge region 408, template copy 411b includes original (parental) template strand 407a (dashed and black) and newly synthesized daughter strand 307b′ (in gray). Further, the double-length DNA 411 template includes a first terminal end 412a and a second terminal end 412b. The first terminal end 412a, for example, includes the portion of the YB-UMI/SID-EA 400 strand 400b associated with 5′ end of nick site 401b YB-UMI/SID-EA 400 (open black rectangle at terminal end 412a) and its copy (open gray circles at terminal end 412a). Likewise, second terminal end 412b of the double-length DNA template 411 includes the portion of the YB-UMI/SID-EA 400 strand 400a associated with 5′ end of nick site 401a YB-UMI/SID-EA 400 (open black circles at 412b) and its copy (open gray rectangle at terminal end 412b).

As is also shown, template copy 411a also includes at terminal end 412a Y-branch element 413b and its complementary sequence in Y-branch element daughter strand 413b′, along with SID 418a and its complementary daughter SID copy 418b. Likewise, on the other end of the double-length DNA template (i.e., terminal end 412b), shown is Y-branch element 413a and its complementary sequence in Y-branch element daughter strand 413a′, along with SID 417a and its complementary daughter SID copy 417b.

In this way, combination of the YB-UMI/SID-EA 400 with a target DNA template strand yields a double-length DNA template 411 that includes a predetermined oligonucleotide sequence at each end (i.e., the Y-branch elements 413a and 413b and their respective complementary copies 413a′ and 413b′), an SID and its complementary copy at each end (i.e., SIDs 418a and 417a and their respective 418b′ and 417b′ complementary copies), and a UMI 416 (and its strand sequences 416a and 416b). And while FIG. 4B shows the location of the SID sequences 418a, 418b′, 417a, and 417b′ contiguous with the Y-branch elements 413b, 413b′, 413a, and 413a′, it is to be understood that that the SIDs can be located at different locations within the double-length DNA template. For example, if no Y-branch elements are present on an end adapter including the SIDs, the resultant double-length DNA template can include the SIDs at the first and second terminal ends 412a and 412b, with no Y-branch elements.

With such strand specific information encoded in each strand of the double-length DNA template 411, the individual strands of the double-length DNA template 411 can easily be identified and differentiated in a multiplex PCR reaction. In fact, because multiple copies of the SIDs will appear in a single, duplicated PCR product strand, bioinformatically the SIDs of the YB-UMI/SID-EA 400 and its resultant double-length DNA template 411 may be determined with high accuracy, thereby reducing or eliminating the need for additional for error correction. The SIDs can also be used as landmarks in a given strand, allowing additional analytics.

### Asymmetric DNA Template Extension & Formation

In certain example embodiments, provided are asymmetric DNA template copies and methods of making the asymmetric DNA template. That is, the methods can compositions provided herein can be used to produce an asymmetric DNA template in which only one strand of the target DNA template is duplicated. Hence, the asymmetric DNA template is an asymmetric DNA template in that only one strand of the parental template is duplicated. Such asymmetric DNA templates find use in sequence preparation work-flows that require a single-stranded DNA molecule as a target template, such as the “Sequencing by Expansion” methodology developed by the inventors (see, e.g., US Published Patent Application No. 20220042075), which is herein incorporated by reference in its entirety.

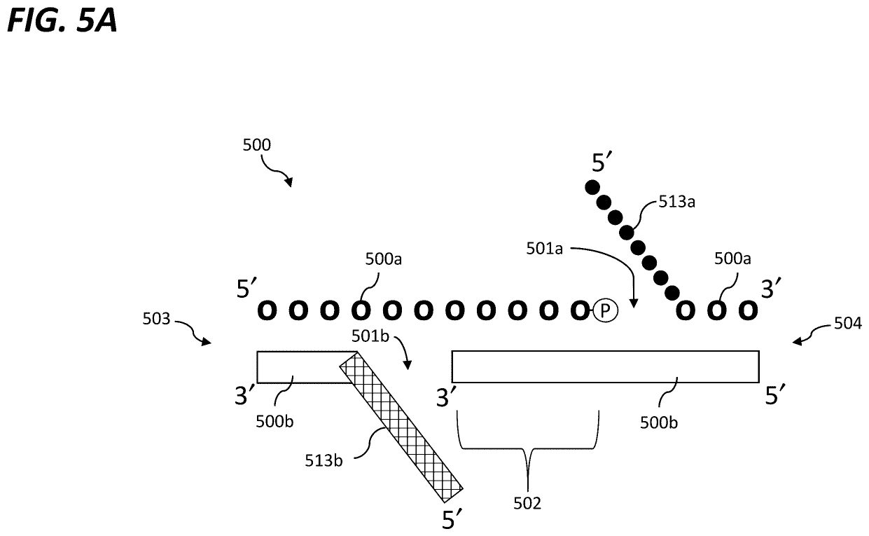

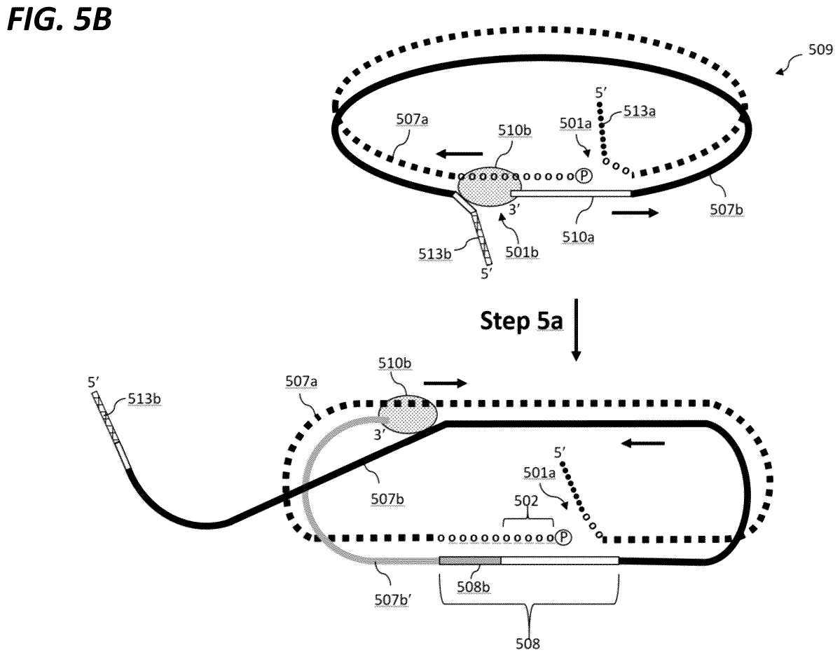

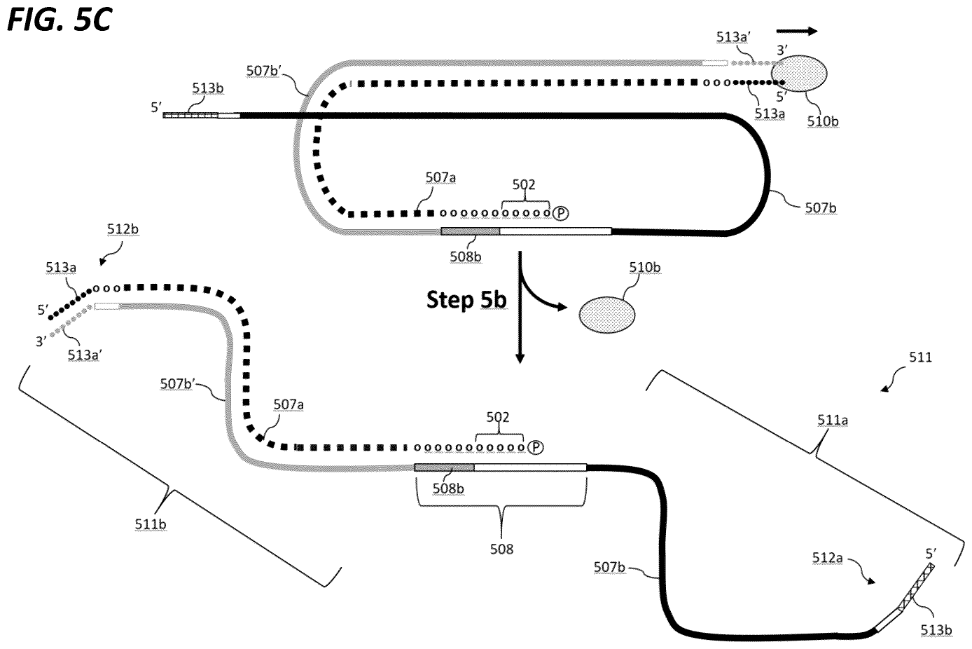

With reference to FIG. 5A, provided is an illustration showing a modified Y-branched end adapter according to FIG. 2A, but that has been modified so that it accommodates only a single polymerase attachment and unidirectional extension, in accordance with certain example embodiments. In other words, the modified Y-branched end adapter (or “modified YBEA”)—when ligated to a target DNA template—accommodates only unidirectional extension of the target DNA template. For example, the modified YBEA 500 includes hybridized strands 500a (circles) and 500b (rectangles), thus forming a polynucleotide duplex. The modified YBEA 500 also has the general EA structure as in FIG. 2A, in that a first Y-branch element 513a and second Y-branch element 513b is added to the 5′ end of the first and second nick sites 501a and 501b, respectively. Further, the modified YBEA 500 includes terminal ends 503 and 504 flanking each nick site 501a and 501b, each terminal end 503 and 504 being compatible with efficient ligation to the ends of a target DNA template as described herein. That is, the terminal ends are ligatable to a target DNA template.

Yet unlike the YBEA 200 of FIG. 2A, 3′ end of one of the nick sites—which provides a polymerase extension site as described herein—is blocked or otherwise modified so as to prevent polymerase binding and/or extension in the modified YBEA. Notably, any method known in the art for modifying a 3′ end such that it prevents polymerase binding and/or extension can be used. For example, the 3′ end can be phosphorylated. As shown in the example modified YBEA 500 of FIG. 5A, 3′ end associated with nick site 501a includes a phosphorylated 3′ end, thus preventing polymerase extension of 3′ nick site 501a as described further herein. And while not shown for simplicity, the modified YBEA 500 can, in certain example embodiments, included a UMI and/or one or more SIDs as described herein.

In certain example embodiments, the modified YBEA—with a single, extendable nick site—can be combined with a target DNA template to form a circular construct. That is, the modified YBEA can be ligated to both ends of a target DNA template, such as is described in FIGS. 2B and 3B. Following ligation, a bridge is formed between the two ends to the target DNA template, with the modified YBEA 500 serving as the bridge between. Once the modified YBEA forms the bridge joining the terminal ends of the target DNA template, a circular construct is formed, such as is described in FIG. 2B (at Steps 2a and 2b) and FIG. 3B (Steps 3a and 3b).

With reference to FIG. 5B, provided is a schematic depicting polymerase attachment and initiation of unidirectional extension of a circular construct, in accordance with certain example embodiments. As shown, the circular construct 509 includes parental strands (in black) 507a (dashed line) and 507b (solid line) of a target DNA template, along with Y-branch elements 513a and 513b. Further, nick site 501a includes a phosphorylated 3′ end, thus preventing polymerase extension of the 3′ end at the 501a nick site. When polymerase 510b is combined with the DNA circular construct 509, however, it binds to nick site 501b—where there is no 3′ modification in this example—to proceed in the direction opposite of nick site 501a (as indicated by the arrow). Polymerase 510b also displaces 5′ end of parental template strand 507b, including displacement of its associated Y-branch element 513b. In the absence of polymerase binding/extension at nick site 501a, however, parental strand 507a remains bound to its complementary strand 507b at the nick site, i.e., there is no displacement of 5′ end of strand 507a as with bidirectional target DNA template extension as described herein.

At Step 5a of FIG. 5B, polymerase 510b continues to unidirectionally extend the circular construct 509 (see arrow). That is, as polymerase 510b extends the 3′ end of nick site 501b while also displacing 5′ end of nick site 501b (and its associated parental template strand 507b), using parental strand 507a as a template (FIG. 5B, lower panel). Hence, as polymerase 510b proceeds at Step 5a (lower panel), it extends 3′ end of nick site 501b using parental strand 507a as a template to synthesize the new, daughter strand 507b′ (in gray) that is complementary to the sequence of parental strand 507a (and that hence shares the same sequence of parental strand 507b). As shown, Y-branch element 513b also remains attached to displaced parental strand 507b at the 5′ end. The new daughter strand 507b′ also includes, as part of the bridge 508, replicated ssDNA daughter strand bridge portion 508b. With its phosphorylated (and hence blocked) 3′ end, however, nick site 501a remains and is not extended.

Continuing with the above example embodiment, FIG. 5C is a schematic depicting continued polymerase extension of the circular construct and formation of the asymmetric template using the modified YBEA 500, in accordance with certain example embodiments. As shown, polymerase 510b continues along parental template strand 507a to 5′ end of parental template strand 507a, completing the synthesis of new daughter strand 507b′. Notably, new daughter strand 507b′ includes a Y-branch element daughter strand 513a′, Y-branch element daughter strand 513a′ being complementary to Y-branch element 513a attached to parental strand 507a. As shown in FIG. 5C, in this example embodiment parental template strand 507b is fully displaced, but this strand is not duplicated because of the blocked 3′ end associated with 501a (see FIG. 5B).

At Step 5b of FIG. 5C, once polymerase 510b completes synthesis of daughter strand 507b′, polymerase 510b dissociates the DNA complex, forming the asymmetric DNA template 511 (lower panel). As illustrated in FIG. 5D (lower panel), the asymmetric DNA template 511 includes parental template strands 507a and 507b, each flanking the bridge 508 (the bridge being derived from the modified YBEA 500 and including bridge daughter strand portion 508b). The symmetric DNA template 511 also includes a first terminal end 512a and a second terminal end 512b. But with the unidirectional extension via polymerase 510b, only a single daughter strand is present, i.e., strand 507b′, in the asymmetric DNA template 511. As shown, the asymmetric portion of the asymmetric DNA template 511 includes the strand 507b′, with Y-branch element 513b located at the 5′ end of the 507b′ strand (at terminal end 512a). Further, the bridge portion 508, with the newly synthesized daughter bridge portion 508b, contiguously and covalently links parental template strand 507b with newly synthesized daughter strand 507b′.

As also shown, template copy 511b includes the daughter strand 507b′, as a complement to parental template strand 507a. Parental strand 507a also includes Y-branch element 513a at its 5′ end, while new daughter strand 507b includes daughter Y-branch element 513a′ at 3′ end. In this way, combination of the modified YBEA 500 with a parental target DNA template to form a circular construct, followed by polymerase-mediated extension as described in Steps 5a and 5b of FIGS. 5B and 5C, form the asymmetric DNA template.

### Multi-Length Template Extension

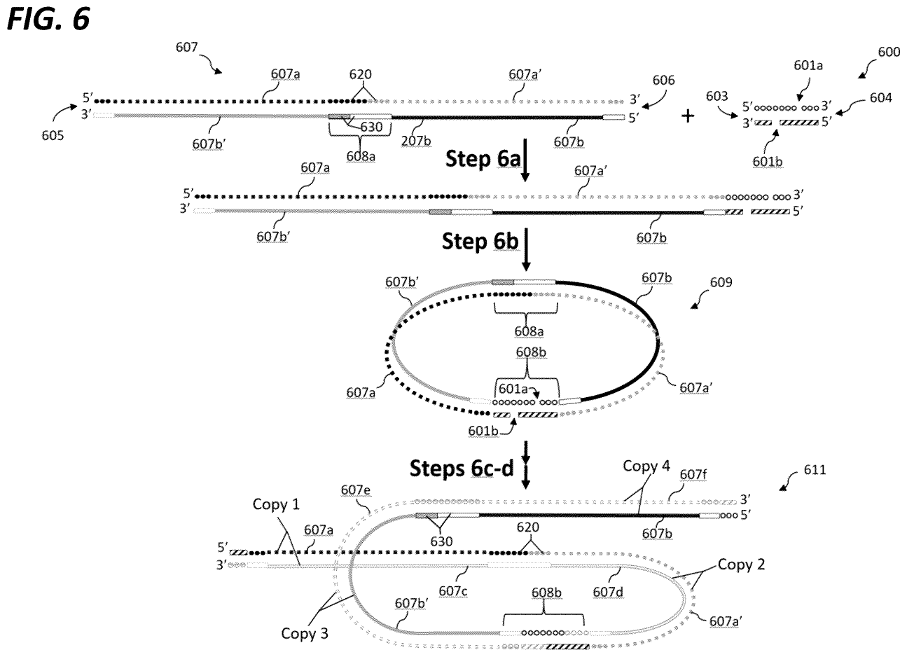

In certain example embodiments, the methods and compositions described herein can be repeated any number of times—starting with the first double-length DNA template—to form a multiple length DNA template. For example, after forming the double-length DNA template according to the methods and compositions described herein, both ends of the double-length DNA template can be ligated to a second end adapter (EA)—the second EA, for example, having the features of the EA of FIG. 1A. This forms a circular construct that includes the double-length DNA template and the ligated EA. Thereafter, the circular construct can be bidirectionally replicated as described herein, forming a quadruple-length DNA template or “double double” template, i.e., a DNA molecule that includes a duplicated copy of the original double-length DNA template. The quadruple-length DNA template, for example, includes the two parental target DNA template strands that arise from the original double-length DNA template, along with their complementary daughter strands as described herein. The quadruple-length DNA template, however, also includes a duplicate of these strands and hence includes four copies of the target DNA template. The formation of such a quadruple length DNA template, for example, is illustrated in FIG. 6.

As shown, the target DNA template of the example in FIG. 6 is a double-length DNA template that includes two copies of the original target DNA template as described herein and hence is referred to in this example as a target double-length DNA template 607. The two copies of the original DNA template, for example, include hybridized polynucleotide strands 607a and 607b′ (the first copy) and hybridized polynucleotide strands 607b and 607a′ (the second copy). And as also described herein, both copies included both parental DNA from the original target DNA template (strands 607a and 607b, shown in black) and their complementary copy strands (strands 607b′ and 607a′ shown in gray). The two copies are separated by a first double-stranded bridge region 608a (i.e., the original bridge) that is derived from the initial (or first) end adapter used to form the target double-length DNA template 607, as described herein. The first bridge 608a, for example, includes strands 620 and 630. The target double-length DNA template 607 also includes first and second template terminal ends 605 and 606, respectively, both of which are ligatable to a second EA 600.

Also shown is the second end adapter (EA) 600, which has the structure, for example, as the EA 100 of FIG. 1A. For example, the second EA 600 include a first nick site 601a and second nick site 601b, both of which can accommodate polymerase binding and extension (e.g., bidirectional extension as described herein). The EA 600 also includes first and second EA terminal ends 605 and 606, respectively. Both EA terminal ends 605 and 606, for example, are ligatable to the target double-length DNA template 607, as described herein.

At Step 6a, for example, the second EA 600 is ligated to either end of the target double-length DNA template 607. For example, terminal end 603 of the second EA 600 is ligated to template end 606 (FIG. 6, Step 6a). Alternatively at Step 6a, and although not shown for simplicity, the other terminal end of the second EA 600 (i.e., end 604) is ligated to terminal end 605 of the target double-length DNA template 607. Either way, one end of the second EA 600 is joined to the end of the target double-length DNA template.

At Step 6b of FIG. 6, the remaining free end of the second EA 600 is ligated to the remaining free end of the target double-length DNA template 607 to form a circular construct 609. That is, at Step 6b of FIG. 6B the two terminal ends 603 and 604 of the second EA 600 join each end 605 and 606 of the target double-length template 607, thereby forming a second DNA bridge 608b between the ends of the target double-length template 607. That is, the entirety of the second EA 600 forms the second DNA bridge 608b between the two ends 605 and 606 of the target double-length DNA template 607. This forms the circular construct 609 that includes the first EA (as bridge 608a) and the second bridge 608b (formed from the second EA 600). In this way, the second EA 600 operates as bridge precursor for the second bridge region 608b of the circular construct 609. The respective first and second nick sites 601a and 601b remain in the circular construct 609 (as part of the second bridge region 608b) and hence, in certain example embodiments, provide two respective 3′ ends available for polymerase attachment and bidirectional extension, as described herein.

At Steps 6c-6d, the circular construct 609 is replicated, such as is described with regard to Steps 1c-1d of FIGS. 1C and 1D (with these Steps combined in FIG. 6 for simplicity). As shown, the completion of Steps 6c-6d of FIG. 6 results in a quadruple-length DNA template 611. For example, at Step 6c, the circular construct 609 is contacted with polymerases (e.g., a first and second polymerase (not shown)) that bind to nick sites 601a and 601b of the circular construct 609. Thereafter, the polymerases bidirectionally extend the circular construct 609. For example, one of the polymerases extends 3′ end of nick site 601a, while also displacing 5′ end of nick site 601a. Likewise, the other polymerase extends 3′ end of nick site 601b, while also displacing 5′ end of nick site 601b. At Step 6d, once the polymerases complete their extension reaction of the circular construct 609, they dissociate from the circular construct 609, forming the quadruple-length DNA template 611.

As shown, the quadruple-length DNA template 611 includes four copies of the target sequence. For example, Copy 1 includes original parental target template DNA strand 607a—as carried through from the original parental target DNA template to the double-length DNA template—and its complementary newly synthesized non-parental strand 607c. Copy two, for example, includes—from the double-length DNA template—non-parental strand 607a′, along with newly synthesized non-parental strand 607d. As shown, Copy 1 and 2 are separated by a strand segment 620 (black and gray circles) of the first bridge 608a, along with its newly synthesized complementary portion (gray rectangle).

Likewise, Copy 3 includes—from the double-length DNA template—non-parental strand 607b′, along with newly synthesized non-parental strand 607e. As shown, Copy 2 and 3 are separated by the second bridge region 608b, the second bridge region 608b including portions form the EA 600 (in black) and newly synthesized portions thereof (in gray). Further, Copy 4 includes original parental target template DNA strand 607b—as carried through from the original parental target DNA template to the double-length DNA template—and its complementary newly synthesized non-parental strand 607f. As shown, Copy 3 and 4 are separated by a strand segment 630 (open and gray boxes) of the first bridge 608a, along with its newly synthesized complementary portion (gray hatch-lined circles).

Notably, in the example of FIG. 6, in 5′→3′ direction polynucleotide parental strand 607a is contiguously joined to—via the bridge region sequences—non-parental strand copies 607a′, 607e, and 607f. Parental strand 607a also shares the same 5′→3′ polynucleotide sequence as non-parental strand copies 607a′, 607e, and 607f. Likewise, in 5′→3′ direction polynucleotide parental strand 607b is contiguously joined to—via the bridge region sequences-non-parental strand copies 607b′, 607d, and 607c. Parental strand 607b also shares the same 5′→3′ polynucleotide sequence as non-parental strand copies 607b′, 607d, and 607c.

While FIG. 6 illustrates the formation of a quadruple-length DNA template using the EA of FIG. 1A, for example, it is to be understood that the method of FIG. 6 can be repeated for multiple iterations, with a doubling of the number of target DNA template copies each time. For example, an initial double-length DNA template include two copies of the target DNA template, as described herein, while an additional duplication—as in the example method of FIG. 6—produces four copies of the target DNA template, i.e., the quadruple-length DNA template 611. Thereafter, additional iterations produce 8, 16, 32, 64, etc. of the initial of target DNA template.

Further, it is to be understood that any of the end adapters described herein, and their associated methods of use, can be used to form a quadruple-length DNA template. Or, when the replication described in FIG. 6 repeated, any of the end adapters described herein, and their associated methods use, can be used to form a multi-length DNA template. This includes, for example, the use of different EAs at different iterations when forming a multi-length DNA template.

For example, the initial double-length DNA template may be formed using the EA of FIG. 1A, with a second iteration also using the EA of FIG. 1A to form the quadruple-length DNA template 611. Thereafter, an additional iteration may use the EA of FIG. 2A (EA 200), FIG. 3A, and/or FIG. 4A (EA 300) to form an 8-copy multi-length DNA template. Hence, in certain example embodiments, the quadruple-length DNA template or the multi-length DNA template can include Y-branched EAs to facilitate subsequent PCR amplification and/or UMI and SIDs to facilitate bioinformation analyses (including genetic and epigenetic analyses as described herein). In fact, such quadruple-length DNA template and the multi-length DNA templates are particularly useful in validation of sequencing reactions and their associated data. Additionally or alternatively, in certain example embodiments the one or more of the EAs can include a protected nick site as described herein (e.g., FIGS. 5A and 5B) to form an asymmetric double- or multi-length DNA template.

### SPECIFICALLY INCLUDED EMBODIMENTS

The following embodiments are specifically contemplated as part of the disclosure. This is not intended to be an exhaustive listing of potentially claimed embodiments included within the scope of the disclosure.
Embodiment 1. A linear end adapter for duplicating a target DNA template, the linear end adapter comprising: a first polynucleotide strand hybridized to a second polynucleotide strand, thereby forming polynucleotide duplex, the polynucleotide duplex comprising a first terminal end and a second terminal end; a first nick site and second nick site, wherein the first nick site is located within the first polynucleotide strand of the polynucleotide duplex and wherein the second nick site is located within the second polynucleotide strand of polynucleotide duplex; and, a spacer region separating the first and second nick sites from each other, thereby linearly offsetting the first nick site from the second nick site.
Embodiment 2. The linear end adapter of embodiment 1, wherein the first nick site of the first polynucleotide strand comprises a discontiguous break in a sequence of the first polynucleotide strand and/or wherein the second nick site of the second polynucleotide strand comprises a discontiguous break in a sequence of the second polynucleotide strand.
Embodiment 3. The linear end adapter of embodiment 1 or 2, wherein each terminal end is configured for ligation to both ends of the target DNA template.
Embodiment 4. The linear end adapter of embodiment 3, wherein the first terminal end and/or the second terminal end of the polynucleotide duplex comprises ligatable blunt ends.
Embodiment 5. The linear end adapter of embodiment 3, wherein the first terminal end and/or the second terminal end of the polynucleotide duplex comprises ligatable blunt ends.
Embodiment 6. The linear end adapter of embodiment 3, wherein the first terminal end and/or the second terminal end of the polynucleotide duplex comprises a ligatable nucleic acid overhang.
Embodiment 7. The linear end adapter of any of embodiments 1-5, wherein each nick site is configured for a polymerase-mediated extension reaction.
Embodiment 8. The linear end adapter of any of embodiments 1-6, wherein the linear offset between the first nick site and the second nick site corresponds to distance that accommodates the binding of a polymerase to the first nick site and to the second nick site.
Embodiment 9. The linear end adapter of any of embodiments 1-7, wherein the first nick site and/or the second nick site comprise a 3′ end and a 5′ end.
Embodiment 10. The linear end adapter of embodiment 8, wherein the linear end adapter further comprises a first Y-branch element sequence attached to the 5′ end flanking the first nick site and/or a second Y-branch element sequence attached to the 5′ end flanking the second nick site.
Embodiment 11. The linear end adapter of embodiment 9, wherein the sequence of the first and/or second Y-branch element encodes a primer binding sequence.
Embodiment 12. The linear end adapter of embodiment 9 or 10 wherein the sequence of the first and/or second Y-branch element is approximately 5-25 nucleotides in length.
Embodiment 13. The linear end adapter of any of embodiments 1-11, wherein the first polynucleotide strand and/or the second polynucleotide strand comprises a unique molecular identifier (UMI) sequence.
Embodiment 14. The linear end adapter of embodiment 12, wherein the UMI is located within the spacer region.
Embodiment 15. The linear end adapter of any of embodiments 1-13, wherein the first polynucleotide strand comprises a first sequence index (SID) and/or wherein the second polynucleotide strand comprises a second SID.
Embodiment 16. The linear end adapter of any of embodiments 9-13, wherein the first polynucleotide strand comprises a first sequence index (SID) and wherein the second polynucleotide strand comprises a second SID, wherein the sequence of the first SID is contiguous with the sequence of the first Y-branch element and wherein the sequence of the second SID is contiguous with the sequence of the second Y-branch element.
Embodiment 17. The linear end adapter of any of embodiments 1-15, wherein the linear end adapter is approximately 50-100 nucleotides in length.
Embodiment 18. The linear end adapter of any of embodiments 1-16, wherein the spacer region is approximately 10-50 nucleotides in length.
Embodiment 19. The linear end adapter of any of embodiments 1-17, wherein the first nick site and/or the second nick site have a length corresponding to approximately 0-10 nucleotides.
Embodiment 20. The liner end adapter of any of embodiments 1-5, wherein only one of the nick sites is configured for a polymerase-mediated extension reaction.
Embodiment 21. The linear end adapter of embodiment 19, wherein either the first nick site or the second nick site comprises a 3′-blocking group that prevents a polymerase-mediated extension reaction.
Embodiment 22. The linear end adapter of embodiment 20, wherein the 3′-blocking group is a phosphate group.
Embodiment 23. The linear end adapter of any of embodiments 19-21, wherein the first nick site and/or the second nick site comprise a 5′ end and wherein the 5′ end of first nick site comprises a first Y-branch element sequence and/or wherein 5′ end of second nick site comprises a second Y-branch element sequence.
Embodiment 24. The linear end adapter of embodiment 22, wherein the first and/or second Y-branch element encode a primer binding sequence.
Embodiment 25. The linear end adapter of any of embodiments 19-23, wherein the spacer region includes a UMI sequence.
Embodiment 26. A method for replicating a target DNA template, the method comprising: performing a ligation reaction between a target DNA template and the linear end adapter according to any of claims 1-24, thereby forming a circular construct, wherein the target DNA template comprises a first target DNA template terminal end and a second the target DNA template terminal end and wherein the ligation reaction (i) joins the first terminal end of the end adapter to the first target DNA template terminal end and (ii) joins the second terminal end of the end adapter to the second target DNA template terminal end, thereby forming the circular construct; and, performing a DNA polymerase-mediated extension reaction of the circular construct, thereby replicating the target DNA template.
Embodiment 27. The method of embodiment 26, wherein performing the DNA polymerase-mediated extension reaction comprises contacting the circular construct with a plurality of strand-displacement polymerases.
Embodiment 28. The method of embodiment 27, wherein the strand displacement polymerase is selected from the group consisting of KAPA HiFi DNA Polymerase, Q5® High-Fidelity DNA Polymerase, and Pfu DNA polymerase, such as a Pfu-X.
Embodiment 29. The method of embodiment 27, wherein the strand displacement polymerase is a phi 29 polymerase.
Embodiment 30. The method of any of embodiments 25-29, wherein the polymerase-mediated extension reaction comprises extension of a 3′ end of the first nick site or a 3′ end of the second nick site of the end adapter.
Embodiment 31. The method of embodiment 30, wherein (i) polymerase-mediated extension of 3′ end of the first nick site of the end adapter or polymerase-mediated extension of 3′ end of second nick site of the end adapter forms an asymmetric DNA template or (ii) wherein polymerase-mediated extension of both 3′ end of the first nick and 3′ end of second nick site of the end adapter forms a double-length DNA template.
Embodiment 32. The method of embodiment 31, wherein a strand of the asymmetric DNA template or the double-length DNA template comprises a unique molecular identifier (UMI).
Embodiment 33. The method of embodiment 32, wherein the UMI is located with a strand of a bridge region of the asymmetric DNA template or the double-length DNA template.
Embodiment 34. The method of any of embodiments 31-33, wherein a strand of the asymmetric DNA template or the double-length DNA template comprises a sequence index (SID).
Embodiment 35. The method of embodiment 34, wherein the SID is located with a strand of a bridge region of the asymmetric DNA template or the double-length DNA template and/or at a terminal end of the asymmetric DNA template or the double-length DNA template.
Embodiment 36. The method of any of embodiments 26-34, where a terminal end of the asymmetric DNA template or the double-length DNA template comprises a Y-branch end adapter.
Embodiment 37. The method of embodiment 36, wherein the Y-branch end adapter encodes a primer binding site.
Embodiment 38. A method of preparing a double-length DNA template a from target DNA template, the method comprising: performing a ligation reaction between a target DNA template and the end adapter according to any of claims 1-18, thereby forming a circular construct, wherein the target DNA template comprises a first target DNA template terminal end and a second target DNA template terminal end and wherein the ligation reaction (i) joins the first terminal end of the end adapter to the first target DNA template terminal end and (ii) joins the second terminal end of the end adapter to the second target DNA template terminal end; and, performing a DNA polymerase-mediated extension reaction of the circular construct, thereby forming a double-length DNA template that comprises a first copy and a second copy of the target DNA template.
Embodiment 39. The method of embodiment 38, wherein performing the DNA polymerase-mediated extension comprises contacting the circular construct with a plurality of strand-displacement polymerases.
Embodiment 40. The method of embodiment 39, wherein the strand displacement polymerase is selected from the group consisting of KAPA HiFi DNA Polymerase, Q5® High-Fidelity DNA Polymerase, and Pfu DNA polymerase, such as a Pfu-X.
Embodiment 41. The method of embodiment 39, wherein the strand displacement polymerase is a phi 29 polymerase.
Embodiment 42. The method of any of embodiments 38-41, wherein the polymerase-mediated extension reaction comprises extension of a 3′ end of the first nick site and a 3′ end of the second nick site of the end adapter.
Embodiment 43. The method of any of embodiments 38-42, wherein the polymerase-mediated extension is bidirectional.
Embodiment 44. The method of any of embodiments 38-42, wherein the first copy of the target DNA template and the second copy of the target DNA template are contiguously joined to each other by a DNA bridge region.
Embodiment 45. The method of embodiment 44, wherein the bridge region is derived from the end adapter.
Embodiment 46. The method of embodiment 44 or 45, wherein each polynucleotide strand of the double-length DNA template comprises a 5′ to 3′ parental strand of the target DNA template and a 5′ to 3′ daughter strand copy of the parental strand of the target DNA template.
Embodiment 47. The method of embodiment 46, wherein the parental strand of the target DNA template and the daughter strand copy of the target DNA template are contiguously joined to each other by a 5′ to 3′ strand of the DNA bridge region.
Embodiment 48. The method of embodiment 47, wherein the strand of the bridge region comprises a unique molecular identifier (UMI).
Embodiment 49. The method of embodiments 47 or 48, wherein the strand of the bridge region comprises a sequence index (SID).
Embodiment 50. The method of any of embodiments 46-49, where the double-length DNA template comprises a first terminal end and a second terminal end and wherein the first terminal end and/or the second terminal end comprise an SID.
Embodiment 51. The method of any of embodiments 38-50, wherein the linear end adapter comprises a first Y-branch element sequence and a second Y-branch element sequence and wherein performing the DNA polymerase-mediated extension reaction positions the first Y-branch element sequence and the second Y-branch sequence at the 5′ end of each parental strand of the double-length DNA template.
Embodiment 52. The method of embodiment 51, wherein the polymerase-mediated extension reaction of the DNA circular construct synthesizes a first daughter Y-branch element sequence and a second daughter Y-branch element sequence, wherein the first daughter Y-branch element sequence is complementary to the first Y-branch element sequence and wherein the second daughter Y-branch element sequence is complementary to the Y-branch element sequence.
Embodiment 53. The method of embodiment 52, wherein the first daughter Y-branch element sequence and the second daughter Y-branch element sequence are located on 3′ end of each daughter strand copy of the double-length DNA template.
Embodiment 54. The method of embodiments 51 or 53, wherein the Y-branch element encodes a primer binding site.
Embodiment 55. The method of any of embodiments 38-54, wherein the method is serially repeated to form a quadruple-length DNA template or a multi-length DNA template.
Embodiment 56. A double-length DNA template formed from the method of any of claims 38-55.
Embodiment 57. A method of identifying epigenetic information associated with a target nucleic acid sequence, comprising: (a) ligating a linear target DNA template to both ends of the linear end adapter according to any of claims 1-18, thereby forming a circular DNA construct; (b) performing a DNA polymerase-mediated bidirectional extension reaction of the circular DNA construct in the presence of a plurality of protected cytosine nucleotides, thereby forming a double-length DNA template that comprises the protected cytosine nucleotides; (c) denaturing the double-length DNA template; (d) subjecting the denatured double-length DNA template to a bisulfite conversion reaction, thereby forming bisulfite-converted double-length DNA template strands of the double-length DNA template; (e) performing a polymerase chain reaction (PCR) amplification of the bisulfite-converted double-length DNA template strands; (f) sequencing the PCR-amplified bisulfite-converted double-length DNA template strands; and (g) identifying, based on the sequencing of the PCR-amplified bisulfite-converted double-length DNA template strands, epigenetic information associated with a target nucleic acid.
Embodiment 58. The method of embodiment 57, wherein each polynucleotide strand of the double-length DNA template of step (b) comprises a parental template strand from the target DNA template and a daughter copy strand of the parental template strand.
Embodiment 59. The method of embodiment 58, wherein the parental template strand is contiguously joined to the daughter copy strand of the parental template strand by a single-stranded bridge region.
Embodiment 60. The method of embodiment 59, wherein the single-stranded bridge region is derived from the end adapter.
Embodiment 61. The method of embodiment 58 or 59, wherein the protected cytosine nucleotides are incorporated into the daughter copy strand of the parental template strand during the DNA polymerase-mediated bidirectional extension reaction of step (b).
Embodiment 62. The method of any of embodiments 58-61, wherein the sequencing of the PCR-amplified bisulfite-converted double-length DNA template strands of step (f) provides a polynucleotide sequence for the parental template strand and the daughter copy strand and wherein identifying the epigenetic information associated with the target nucleic acid comprises an intra-strand comparison of the polynucleotide sequence of the parental template strand with the polynucleotide sequence of the daughter copy strand.
Embodiment 63. The method of embodiment 62, wherein a sequence discrepancy location between the polynucleotide sequence of the parental template strand and the polynucleotide sequence of the daughter copy strand identifies an unprotected cytosine residue location in the parental template strand.
Embodiment 64. The method of embodiment 63, wherein the unprotected cytosine residue location in the parental template strand corresponds to an unprotected cytosine residue location in the target nucleic acid sequence.
Embodiment 65. The method of any of embodiments 62-64, wherein a cytosine residue location in the sequence of the parental template strand indicates a corresponding location of a protected cytosine in the target nucleic acid sequence.
Embodiment 66. The method of embodiment 57, wherein the double-length DNA template of step (b) comprises a first copy and a second copy of the target DNA template.
Embodiment 67. The method of embodiment 66, wherein the first copy and the second copy of the target DNA template are joined together by a double-stranded bridge region.
Embodiment 68. The method of embodiment 67, wherein the double-stranded bridge region is derived from the end adapter.
Embodiment 69. The method of any of embodiment 66-68, wherein each copy of the target DNA template within the double-length DNA template comprises a parental template strand and a daughter strand that is complementary and hybridized to the parental template strand.
Embodiment 70. The method of embodiment 69, wherein the protected cytosine nucleotides are incorporated into the hybridized complementary daughter strand during the DNA polymerase-mediated bidirectional extension reaction of step (b).
Embodiment 71. The method of embodiment 69 or 70, wherein sequencing the PCR-amplified bisulfite-converted double-length DNA template strands of step (f) provides a polynucleotide sequence for the parental template strand and its hybridized complementary daughter strand and wherein identifying the epigenetic information associated with the target nucleic acid comprises an inter-strand comparison of the polynucleotide sequence of the parental template strand with the polynucleotide sequence of the hybridized complementary daughter.
Embodiment 72. The method of embodiment 71, wherein a nucleotide mismatch location between the polynucleotide sequence of the parental template strand and the hybridized complementary daughter identifies an unprotected cytosine residue location in parental template strand.
Embodiment 73. The method of embodiment 72, wherein the unprotected cytosine residue location in the parental template strand corresponds to an unprotected cytosine residue location in the target nucleic acid sequence.
Embodiment 74. The method of any of claims 57-73, wherein the protected cytosine nucleotides comprise methylated cytosine residues.
Embodiment 75. The method of any of embodiments 57-73, wherein the unprotected cytosine nucleotides are unmethylated cytosine residues.
Embodiment 76. The method of any of embodiments 57-75, wherein the double-length DNA template of step (b) comprises a unique molecular identifier (UMI).
Embodiment 77. The method of embodiment 76, wherein the UMI is located in the single-stranded bridge region of embodiment 60 or the double-stranded bridge region of embodiment 68.
Embodiment 78. The method of any of embodiments 57-77, wherein the double-length DNA template of step (b) comprises a sequencing index (SID).
Embodiment 79. A double-length DNA template, the double-length DNA template comprising a first copy and a second copy of target DNA template, wherein the first copy and the second copy of the target DNA template are contiguously joined to each other by a double-stranded bridge region.
Embodiment 80. The double-length DNA template of embodiment 79, wherein each polynucleotide strand of the double-length DNA template comprises a parental template strand from the target DNA template and a daughter copy strand of the parental template strand.
Embodiment 81. The double-length DNA template of claim 79 or 80, wherein the parental template strand is contiguously joined to the daughter copy strand of the parental template strand by a strand of the bridge region.
Embodiment 82. The double-length DNA template of embodiment 79, wherein each copy of the target DNA template within the double-length DNA template comprises a parental template strand and a daughter strand that is complementary and hybridized to the parental template strand.
Embodiment 83. The double-length DNA template of any of embodiments 79-82, wherein the double-length DNA template comprises a first terminal end and a second terminal end, wherein either terminal end comprises a sequence encoding a primer binding site.
Embodiment 84. The double-length DNA template of any of embodiments 79-83, wherein the bridge region or a strand thereof includes a unique molecular identifier (UMI).

What is claimed is:

1. A linear end adapter for duplicating a target DNA template, the linear end adapter comprising:
a first polynucleotide strand hybridized to a second polynucleotide strand, thereby forming polynucleotide duplex, the polynucleotide duplex comprising a first terminal end and a second terminal end;
a first nick site and second nick site, wherein the first nick site is located within the first polynucleotide strand of the polynucleotide duplex and wherein the second nick site is located within the second polynucleotide strand of polynucleotide duplex; and,
a spacer region separating the first and second nick sites from each other, thereby linearly offsetting the first nick site from the second nick site.

2. The linear end adapter of claim 1, wherein the first nick site of the first polynucleotide strand comprises a discontiguous break in a sequence of the first polynucleotide strand and/or wherein the second nick site of the second polynucleotide strand comprises a discontiguous break in a sequence of the second polynucleotide strand.

3. The linear end adapter of claim 1, wherein each terminal end is configured for ligation to both ends of the target DNA template.

4. The linear end adapter of claim 3, wherein the first terminal end and/or the second terminal end of the polynucleotide duplex comprises ligatable blunt ends.

5. The linear end adapter of claim 3, wherein the first terminal end and/or the second terminal end of the polynucleotide duplex comprises a ligatable nucleic acid overhang.

6. The linear end adapter of claim 1, wherein each nick site is configured for a polymerase-mediated extension reaction.

7. The linear end adapter of claim 1, wherein the linear offset between the first nick site and the second nick site corresponds to distance that accommodates the binding of a polymerase to the first nick site and to the second nick site.

8. The linear end adapter of claim 1, wherein the first nick site and/or the second nick site comprise a 3′ end and a 5′ end.

9. The linear end adapter of claim 8, wherein the linear end adapter further comprises a first Y-branch element sequence attached to the 5′ end flanking the first nick site and/or a second Y-branch element sequence attached to the 5′ end flanking the second nick site.

10. The linear end adapter of claim 9, wherein the sequence of the first and/or second Y-branch element encodes a primer binding sequence.

11. The linear end adapter of claim 9, wherein the sequence of the first and/or second Y-branch element is approximately 5-25 nucleotides in length.

12. The linear end adapter of claim 1, wherein the first polynucleotide strand and/or the second polynucleotide strand comprises a unique molecular identifier (UMI) sequence.

13. The linear end adapter of claim 12, wherein the UMI is located within the spacer region.

14. The linear end adapter of claim 1, wherein the first polynucleotide strand comprises a first sequence index (SID) and/or wherein the second polynucleotide strand comprises a second SID.

15. The linear end adapter of claim 9, wherein the first polynucleotide strand comprises a first sequence index (SID) and wherein the second polynucleotide strand comprises a second SID, wherein the sequence of the first SID is contiguous with the sequence of the first Y-branch element and wherein the sequence of the second SID is contiguous with the sequence of the second Y-branch element.

16. The linear end adapter of claim 1, wherein the linear end adapter is approximately 50-100 nucleotides in length.

17. The linear end adapter of claim 1, wherein the spacer region is approximately 10-50 nucleotides in length.

18. The linear end adapter of claim 1, wherein the first nick site and/or the second nick site have a length corresponding to approximately 0-10 nucleotides.

19. The liner end adapter of claim 1, wherein only one of the nick sites is configured for a polymerase-mediated extension reaction.

20. The linear end adapter of claim 19, wherein either the first nick site or the second nick site comprises a 3′-blocking group that prevents a polymerase-mediated extension reaction.

21. The linear end adapter of claim 20, wherein 3′-blocking group is a phosphate group.

22. The linear end adapter of claim 19, wherein the first nick site and/or the second nick site comprise a 5′ end and wherein 5′ end of first nick site comprises a first Y-branch element sequence and/or wherein 5′ end of second nick site comprises a second Y-branch element sequence.

23. The linear end adapter of claim 22, wherein the first and/or second Y-branch element encode a primer binding sequence.

24. The linear end adapter of claim 19, wherein the spacer region includes a UMI sequence.


SEQUENCE LISTING (4 sequences)

SEQ ID NO: 1 - DNA, 10 bp, synthetic construct
  source 1..10: mol_type = other DNA
tacacgacgc

SEQ ID NO: 2 - DNA, 10 bp, synthetic construct
  source 1..10: mol_type = other DNA
gcgtcgtgta

SEQ ID NO: 3 - DNA, 10 bp, synthetic construct
  source 1..10: mol_type = other DNA
tacatgacgc

SEQ ID NO: 4 - DNA, 10 bp, synthetic construct
  source 1..10: mol_type = other DNA
cgcagcacat